(19) United States
(12) Patent Application Publication
(10) Pub. No.: US 2016/0199407 A1
(43) Pub. Date: Jul. 14, 2016

(54) HALIDES IN THE TREATMENT OF PATHOGENIC INFECTION

(71) Applicant: THE UNIVERSITY OF IOWA RESEARCH FOUNDATION, Iowa City, IA (US)

(72) Inventors: Botond BANFI, North Liberty, IA (US); Anthony FISCHER, Iowa City, IA (US); Joseph ZABNER, Iowa City, IA (US); Lakshmi DURAIRAJ, Iowa City, IA (US); Daniel LORENTZEN, North Liberty, IA (US); Paul B. McCRAY, JR., Iowa City, IA (US)

(73) Assignee: THE UNIVERSITY OF IOWA RESEARCH FOUNDATION, Iowa City, IA (US)

(21) Appl. No.: 14/972,201

(22) Filed: Dec. 17, 2015

Related U.S. Application Data
(63) Continuation of application No. 12/351,487, filed on Jan. 9, 2009, now abandoned.
(60) Provisional application No. 61/023,724, filed on Jan. 25, 2008.

(51) Int. Cl. A61K 33/18; A61K 45/06; A61M 15/00; A61K 33/00

(52) U.S. Cl. CPC A61K 33/18; A61K 33/00; A61K 45/06; A61M 15/009

(57) ABSTRACT

The present invention relates to the use of halides and halide salts for the treatment of microbial infections, including those caused by bacteria, fungi and viruses. The present invention takes advantage of endogenous immune function and augments this system using a non-toxic and inexpensive reagent that can be delivered to mucosal surfaces, for example, orally, topically, opthalmically and via inhalation.

This application is a continuation application of U.S. application Ser. No. 12/351,487, filed Jan. 9, 2009, which claims benefit of priority to U.S. Provisional Application Ser. No. 61/023,724, filed Jan. 25, 2008, the entire contents of each of the applications being hereby incorporated by reference.

This invention was made with government support under grant nos. N01-AI30040 and PO1 AI060699 awarded by National Institutes of Health. The government has certain rights in the invention.

### BACKGROUND OF THE INVENTION

1. Field of the Invention

The present invention relates generally to the fields of molecular biology and microbiology. More particularly, it concerns the use of halides for the treatment of microbial disease, including those cause by viral and bacterial infections.

2. Description of Related Art

Pathogenic infections (bacterial, fungal, viral) continue to be a major cause of disease in the world, with many causing significant mortalities, as well as contributing substantially to health care costs. For example, influenza virus typically results in 8 million cases of severe illness and up to 500,000 deaths worldwide yearly, which by some definitions is an annual influenza epidemic. Although the incidence of influenza can vary widely between years, approximately 36,000 deaths and more than 200,000 hospitalizations are directly associated with influenza every year in America. Every ten to twenty years a pandemic occurs, which infects a large proportion of the world's population, and can kill tens of millions of people.

Another devasting pathogen-based disease results from methicillin-resistant Staphylococcus aureus (MRSA), referred to as multiply-resistant Staphylococcus aureus or oxacillin-resistant Staphylococcus aureus (ORSA). The organism is often sub-categorized as Community-Associated MRSA (CA-MRSA) or Hospital-Associated MRSA (HA-MRSA) depending upon the circumstances of acquiring disease, based on current data that these are distinct strains of the bacterial species. This organism has evolved an ability to survive treatment with a host of powerful drugs, including penicillin, methicillin, and cephalosporins. MRSA is especially troublesome in hospital-associated (nosocomial) infections. These and other infectious lung disease remain significant health concerns, and new and improved therapies are desperately needed.

### SUMMARY OF THE INVENTION

Thus, in accordance with the present invention, there is provided a method of treating or preventing a viral infection in a subject comprising administering a therapeutically effective amount of a halide. Administering may comprise inhalation, topical administration, oral administration or systemic administration. The subject may be a human, a non-human primate, a dog, a cow, a cat, a horse, a pig, a sheep, a goat, a rabbit, a mouse, a rat, a ferret, a deer, an elk, a bison, a chicken, a turkey, or a parrot. The halide may be iodide or a potassium or sodium salt thereof. Treating may comprise limiting the duration or severity of symptoms, limiting viral replication, decreasing viral load or increasing viral clearance.

The viral infection may be of the lung and/or respiratory system. The subject may be a human that suffers from cystic fibrosis. The viral infection may be caused by respiratory syncytial virus, influenza virus, adenovirus, measles virus, arenavirus, filovirus, echovirus, parainfluenza virus, rhinovirus, Coxsackie virus, Epstein Barr virus, or cytomegalovirus. The viral infection may be caused by a coronavirus or herpesvirus. The method may further comprise administering lactoperoxidase, myeloperoxidase, horseradish peroxidase or an anti-viral drug to the subject.

In another embodiment, there is provided a method of treating or preventing a lung/respiratory pathogen infection in a subject comprising administering a therapeutically effective amount of a halide. The respiratory pathogen may be a bacterium or fungus, such as H. influenzae or S. aureus. The method may further comprise administering lactoperoxidase, myeloperoxidase, horseradish peroxidase or an antibiotic. Treating may comprise decreasing the bacterial load. Administering may comprise inhalation, topical administration, oral administration or systemic administration. The subject may be a human, a domesticated pet or farm animal, a non-human primate, a dog, a cow, a cat, a horse, a pig, a sheep, a goat, a rabbit, a mouse, a rat, a ferret, a deer, an elk, a bison, a chicken, a turkey, or a parrot. The human subject may suffer from cystic fibrosis. The halide may be iodide or a potassium or sodium salt thereof.

In yet another embodiment, there is provided a method of enhancing endogenous respiratory antiviral defense in a subject comprising administering a therapeutically effective amount of a halide.

Another embodiment comprises an inhaler device that delivers a unit dose comprising a therapeutically effective amount of halide or halide salt in a liquid or aerosol carrier. The halide or halide salt may be iodine, NaI, or KI.

It is contemplated that any method or composition described herein can be implemented with respect to any other method or composition described herein.

The use of the word “a” or “an” when used in conjunction with the term “comprising” in the claims and/or the specification may mean “one,” but it is also consistent with the meaning of “one or more,” “at least one,” and “one or more than one.”

The word “about” means plus or minus 5% of the stated number.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

Other objects, features and advantages of the present invention will become apparent from the following detailed description. It should be understood, however, that the detailed description and the specific examples, while indicating specific embodiments of the invention, are given by way of illustration only, since various changes and modifications within the spirit and scope of the invention will become apparent to those skilled in the art from this detailed description.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIG. 1—The oxidative host defense system functions at the apical side of airway epithelia. Duox enzymes are the H₂O₂-generating cytochromes in the apical membrane of airway epithelia. SCN⁻ or I⁻ (depending on their respective concentrations) can be utilized by LPO for OSCN⁻ or HOI generation. LPO is secreted by submucosal glands. HOI and OSCN− have different antimicrobial spectra.

FIGS. 2A-C—Immunostaining of human tracheal submucosa (FIGS. 2A, 2B) and mucosa (FIG. 2C) with anti-NIS antibody photographed at low (FIG. 2A, ×10) and high magnifications (FIG. 2B, 2C, ×63). The first column shows the confocal microscopy images of fluorescent signals, the second column shows the dicroic interference contrast (DIC) images, the third column is the merge of the previous two images. ‘M’ indicates mucous submucosal glands. Serous glands were strongly labeled with the anti-NIS antibody (FIGS. 2A, 2B), whereas the surface epithelium displayed only low level of NIS signal (FIG. 2C).

FIG. 3—Ion chromatography separation and conductivity detection of anions in a human nasal airway surface fluid sample. Nasal airway fluid was collected with a micro-capillary probe (insert) Arrowhead indicates the SCN− peak, “Cl⁻’ indicates the basis of the chloride peak. The SCN⁻ signal was quantified (540 μM) using standard solutions. Arrow indicates the place of I⁻ peak (I⁻ is normally not present in the airway fluid).

FIGS. 4A-B—SCN⁻ (FIG. 4A) and Cl⁻ (FIG. 4B) concentrations in the nasal airway surface liquid (ASL) and serum samples of 10 adult healthy human subjects as determined using anion-echange chromatography. Horizontal lines indicate mean concentrations. Serum and nasal ASL Cl⁻ concentrations and the serum SCN⁻ concentration are in the range of previously published values. SCN⁻ concentration in the nasal ASL has not been reported.

FIGS. 5A-B—Anion-exchange chromatography detection of I⁻ in the nasal ASL without (FIG. 5A) and with (FIG. 5B) iodide supplementation. Arrows indicate the position of the I⁻ peak.

FIG. 6—I⁻ enhances the S. aureus killing activity of airway epithelial cells in the presence of airway surface fluid component LPO (7 μg/ml). Bacterial survival was measured after incubating 1000 CFU S. aureus on the mucosal surface of airway epithelial cells in the presence of the physiological LPO concentration (7 μg/ml) and the indicated concentrations of SCN⁻ (left panel) and I⁻ (right panel). Numbers indicate concentrations in μM. No surviving bacteria was detected in the presence of 50 and 200 μM I⁻, LPO (7 μg/ml) and epithelial cells after 3 hrs. Dotted lines indicate the initial inoculum size.

FIG. 7—Airway epithelial cells kill H. influenzae (H. flu) in the presence of LPO (a physiological airway surface fluid component) and I⁻. Numbers indicate concentrations in μM. No surviving bacteria was detected in the presence of 200 and 500 μM I−, LPO (7 μg/ml) and epithelial cells after adding 1000 CFU bacteria to the mucosal surface for 3 hrs. Dotted line indicates the initial inoculum size, “Cat.” indicates samples with 150 U/ml catalase. The last bar shows that SCN− cannot replace I⁻ in the oxidative mechanism eliminating H. influenzae.

FIG. 8—Microarray expression analysis heatmap. DUOX2 expression is induced (yellow) in human airway epithelia by pro-inflammatory cytokines. Results from 7 different human donor samples are shown. Samples were treated×24 hr, then RNA isolated and microarray hybridization performed using an Affymetrix array (HsAirway). Yellow represents transcripts with increased expression. Blue represents control conditiona. DUOX2 was among most induced transcripts.

FIG. 9—DUOX2 expression is induced in human airway epithelia by RSV A2 strain infection or by IFN-γ. Results from 4 different human donor samples are shown. PBS serves as a negative control. Relative expression data are expressed as mean±SE, n=4.

FIGS. 10—A2 strain of RSV was exposed cell free to the indicated conditions for 5 min, then applied to A549 cells for titering. Y axis indicates the change in titer in log scale.

FIG. 11—Effect of test solution pH on antiviral activity of OSCN and HOI. A2 strain of RSV was exposed cell free to the indicated conditions for 5 min, then applied to A549 cells for titering. Y axis indicates the change in titer in log scale.

FIG. 12—In vitro inactivation of adenovirus. Adenovirus expressing eGFP was exposed cell free to the indicated conditions for 5 min, then applied to A549 cells. 24 hr later, virucidal effects were assessed by relative levels of eGFP in transduced cells. A dose-dependent decrease in GFP expression was seen in the presence of HOI, but not with OSCN⁻.

FIG. 13—In situ inactivation of adenovirus on AEC. Fifty MOI of adenovirus expressing eGFP was added to the apical surface of primary air liquid interface cultures of porcine airway epithelia. Epithelia were treated with ATP (100 μM), 6.5 mg/mL LPO, and the indicated concentration of NaI in a 50 μl vol of PBS, pH 6.5. A dose dependent decrease in GFP expression was seen in the presence of HOI.

FIGS. 14A-D—In situ inactivation of SARS-CoV on airway epithelia. Five MOI of SARS-CoV (Urbani strain) was added to the apical surface of primary air liquid interface cultures of human airway epithelia. Epithelia were treated with ATP (100 μM), 6.5 mg/mL LPO, and the indicated concentration of NaI in a 50 μl of PBS, pH 6.5. 24 hr later, cells were fixed and immunostained for the SARS-CoV N gene product (green staining) and viewed en face by confocal microscopy. HOI treated cells showed a marked reduction immunoreactivity (FIGS. 14A, 14B) compared to untreated control (FIG. 14C). FIG. 14D represents unifected control cells. Red staining indicate cell nuclei.

FIG. 15—Inactivation of A/PR/8/34 on AEC. Twenty MOI of influenza was added to the apical surface of primary air liquid interface cultures of human airway epithelia. Epithelia were treated with ATP (100 μM), 6.5 mg/ml LPO, and 500 μM NaSCN or NaI in a 50 μl of PBS, pH 6.5. En face confocal microscope images show a dose dependent decrease in viral antigen (NS1, green) was seen in the presence of OSCN− (not shown) or HOI. Blue stain indicates nuclei.

FIG. 16. Rates of HOI generation by AEC determined with the HOI probe fluoresceinate. HOI production measured in the presence of apical ATP (to maximize H₂O₂ production) and the indicated combinations of LPO, I⁻, and catalase.

FIGS. 17A-C. Accumulation of I⁻ in the airway surface fluid following oral intake of KI. Airway surface fluid and serum samples were collected before and after ingestion of a KI tablet (130 mg) in a human subject study. Anion composition of the airway fluid and serum samples was analyzed using ion-exchange chromatography. (FIG. 17A) A typical airway fluid chromatogram before KI intake. (FIG. 17B) A typical airway fluid chromatogram 2 hours after KI intake. Arrows indicate the retention time of I⁻. Brackets indicate the Cl⁻ peak (i.e., internal control). (FIG. 17C) I⁻ concentration in the airway surface fluid (triangles) and serum (squares) following KI intake (at the 0 time point). These results indicate that intake of 130 mg KI is more than sufficient to achieve the airway surface fluid I⁻ level (10-50 μM) necessary for the HOI-mediated elimination of bacteria and viruses by Duox and LPO.

FIG. 18. HOI and OSCN⁻ mediated killing of M. haemolytica by AEC. M. haemolytica was incubated on the apical surface of AEC cultures for 3 hours in the presence of indicated compounds. Numbers show concentrations in μM. No surviving bacteria were detected in the presence of LPO and 250 μM I⁻. SCN⁻ also supported killing of M. haemolytica at 250 μM concentration. Dotted line shows the number of inoculated bacteria.

### DESCRIPTION OF ILLUSTRATIVE EMBODIMENTS

### I. DUOX AND THE PRESENT INVENTION

Although numerous antibiotic agents are available for the treatment of bacterial, fungal and viral infections, new treatments are desperately needed to address increasing health care issues such as drug-resistant organisms, new pathogen strains, and high cost of traditional pharmaceuticals. Therefore, in order to maintain the present standards of public health and to limit growing health care costs, new methods of controlling such infections must be devised.

It has been known for years, primarily from literature related to milk and salivary secretions, that there is a host defense system involving oxidation of halide and pseudohalide ions such as thiocyanate (SCN⁻) and iodine (I⁻). For example, thiocyanate can be oxidized by H₂O₂, in a reaction catalyzed by lactoperoxidase (LPO), to produce OSCN(⁻) (hypothiocyanite), an inorganic molecule with antimicrobial activity. In the airways, hydrogen peroxide is generated by a NAPDH oxidase-related protein called Dual Oxidase (DUOX 1 and DUOX2).

The following references are relevant to the action of DUOX: U.S. Pat. No. 6,702,998; U.S. Pat. No. 5,503,853, EP 0 361 908; Pedemonte et al. (2007); Conner et al. (2007); Forteza et al. (2005); Fragoso et al. (2004); Wijkstrom-Frei et al. (2003); Conner et al. (2002); El-Chemaly et al. (2003); and Salathe et al. (1997).

The present inventors have discovered that DUOX2 is an abundant gene product in human airway epithelia following stimulation with pro-inflammatory cytokines. Recently, Moskwa et al. (2007) showed that this host defense system is robust in airway epithelia, and that it is defective in the disease cystic fibrosis (CF). Interestingly, one route for the transcellular transport of halide anions is via CFTR, the gene product that is defective in cystic fibrosis, thereby limiting the availability of one of the components of the host defense system (Moskwa et al., 2007). This work described the anti-bacterial properties of this system against Pseudomonas aeruginosa and Staphylococcus aureus.

The inventors considered these studies, and drew from their teachings the possibility that since DUOX constitutively produces H₂O₂ in the airways, and LPO is secreted from submucosal glands and available in respiratory secretions, the limiting factor in the reaction could be the availability of a halide or pseudohalide, which could be supplied topically to augment defenses. As demonstrated in the Examples below, the delivery of halides, as opposed to pseudohalides, provides an effective method for attacking pathogens in vivo, particular in the context of respiratory tract infections.

Thus, the present invention, taking advantage of the recently defined DUOX host defense system at mucosal surfaces of the airways, encompasses methods to inhibit bacterial, fungal and viral infection through the use of halides and halide salts. This approach offers a simple and cost effective means to supplement and improve existing host defenses systems, creating a powerful, non-specific and broad spectrum treatment for pathogenic disease. Various embodiments are set forth in the following detailed description of the invention.

### II. HALIDES

A. Halides and Halide Salts

A halide is a binary compound of which one part is a halogen atom and the other part is an element or radical that is less electronegative than the halogen, to make a fluoride, chloride, bromide, iodide, or astatide compound. Many salts are halides. All Group 1 metals form halides with the halogens and they are white solids.

A halide ion is a halogen atom bearing a negative charge. The halide anions are fluoride (F⁻), chloride (Cl⁻), bromide (Br⁻), iodide (I⁻) and astatide (At⁻). Such ions are present in all ionic halide salts. Halide salts include sodium chloride (NaCl), potassium chloride (KCl), potassium iodide (KI), lithium chloride (LiCl), copper(II) chloride (CuCl₂), chlorine fluoride (ClF), bromomethane (CH₃Br), iodoform (CHI₃) and silver chloride (AgCl).

B. Pseudohalides

Pseudohalides resemble halides in their charge and reactivity, but are distinct. Common examples are azides (NNN⁻), isocyanate (NCO⁻), isocyanide (CN—), thiocyanate (SCN⁻), etc.

C. Preparations

It is envisioned that the anti-microbial compositions of the present invention can be formulated and administered in virtually any pharmacologically acceptable form, such as parenteral, topical, aerosal, liposomal, nasal or ophthalmic preparations. In those situations, it would be clear to one of ordinary skill in the art the types of diluents and excipients that would be properly used in conjunction with the agents of the present invention. The phrases “pharmaceutically” or “pharmacologically acceptable” refer to molecular entities and compositions that do not produce adverse, allergic, or other untoward reactions when administered to an animal or a human. Thus, as used herein, “pharmaceutically acceptable carrier” includes any and all solvents, dispersion media, coatings, isotonic and absorption delaying agents and the like. Supplementary active ingredients also can be incorporated into the compositions. Solutions of the active compounds as free base or pharmacologically acceptable salts can be prepared in water suitably mixed with a surfactant, such as hydroxypropylcellulose. Dispersions can also be prepared in glycerol, liquid polyethylene glycols, and mixtures thereof and in oils. Under ordinary conditions of storage and use, these preparations contain a preservative to prevent the growth of microorganisms.

Administration of compositions according to the present invention will be via any common route so long as the target tissue is available via that route. This includes oral, nasal, opthalmic, buccal, rectal, vaginal or topical. The active compounds may also be administered parenterally or intraperitoneally. Alternatively, administration may be by orthotopic, intradermal, subcutaneous, intramuscular, intraperitoneal or intravenous injection. The pharmaceutical forms suitable for injectable use include sterile aqueous solutions or dispersions and sterile powders for the extemporaneous preparation of sterile injectable solutions or dispersions. In all cases the form must be sterile and must be fluid to the extent that easy syringability exists. It must be stable under the conditions of manufacture and storage and must be preserved against the contaminating action of microorganisms, such as bacteria and fungi. The carrier can be a solvent or dispersion medium containing, for example, water, ethanol, polyol (e.g., glycerol, propylene glycol, and liquid polyethylene glycol, and the like), suitable mixtures thereof, and vegetable oils. The proper fluidity can be maintained, for example, by the use of a coating, such as lecithin, by the maintenance of the required particle size in the case of dispersion and by the use of surfactants. The prevention of the action of microorganisms can be brought about by various antibacterial an antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin. Sterile injectable solutions are prepared by incorporating the active compounds in the required amount in the appropriate solvent with various of the other ingredients enumerated above, as required, followed by filtered sterilization.

For oral administration, the compositions of the present invention may be incorporated with excipients and used in the form of non-ingestible mouthwashes and dentifrices. A mouthwash may be prepared incorporating the active ingredient in the required amount in an appropriate solvent, such as a sodium borate solution (Dobell's Solution). Alternatively, the active ingredient may be incorporated into an antiseptic wash containing sodium borate, glycerin and potassium bicarbonate.

### III. THERAPEUTIC USES

This invention encompasses methods to reduce virus growth, infectivity, burden, shed, development of anti-viral resistance, and to enhance the efficacy of traditional anti-viral therapies. An attractive feature of these peptides is their tolerance for high salt concentrations. The peptides maintain activity in physiological salt solutions.

A. Bacterial Infections

i. Staphylococcus

Within the family Micrococcaceae, the human pathogenic genus Staphylococcus can be separated from the nonpathogenic genus Micrococcus by various tests, including (1) anaerobic acid production from glucose, (2) sensitivity to 200 μg/ml lysostaphin or to 100 μg furazolidone, and (3) production of acid from glycerol in the presence of 0.4 μg/ml erythromycin, all these tests being positive in the case of staphylococci. Further subclassification into the three main species is of considerable clinical importance (i.e., S. aureus, Staphylococcus epidermidis, and Staphylococcus saprophyticus).

Once the Staphylococcus has been differentiated as Staphylococcus aureus, it is necessary to determine if the S. aureus is methicillin resistant. Older methods such as resistance phenotype, bacteriophage typing, immunoserology, and serotyping of coagulase can be used to type S. aureus. More recently, these methods have been replaced by electrophoretic protein typing, multilocus enzyme electrophoresis, and various genetic techniques, including plasmid analysis, restriction endonuclease analysis of chromosomal DNA, restriction fragment length polymorphisms, ribotyping, nucleotide sequence analysis, and many others.

ii. Bacillus

Bacillus species are rod-shaped, endospore-forming aerobic or facultatively anaerobic, Gram-positive bacteria; in some species cultures may turn Gram-negative with age. The many species of the genus exhibit a wide range of physiologic abilities that allow them to live in every natural environment. Only one endospore is formed per cell. The spores are resistant to heat, cold, radiation, desiccation, and disinfectants. Bacillus anthracis needs oxygen to sporulate; this constraint has important consequences for epidemiology and control. In vivo, B. anthracis produces a polypeptide (polyglutamic acid) capsule that protects it from phagocytosis. The genera Bacillus and Clostridium constitute the family Bacillaceae. Species are identified by using morphologic and biochemical criteria.

The virulence factors of B. anthracis are its capsule and three-component toxin, both encoded on plasmids. B. cereus produces numerous enzymes and aggressins. The principal virulence factors are a necrotizing enterotoxin and a potent hemolysin (cereolysin). Emetic food poisoning probably results from the release of emetic factors from specific foods by bacterial enzymes.

iii. Mycobacterium

Both leprosy and tuberculosis, caused by Mycobacterium leprae and Mycobacterium tuberculosis respectively, have plagued mankind for centuries. With the emergence of antibiotic resistant strains of tuberculosis, research into Mycobacteria has become all the more important in combating these modern mutants of ancient pathogens.

Both the genomes of Mycobacterium tuberculosis and Mycobacterium leprae have been sequenced with hopes of gaining further understanding of how to defeat the infamously successful pathogens. The genome of M. tuberculosis is 4,411,522 base pairs long with 3,924 predicted protein-coding sequences, and a relatively high G+C content of 65.6%. At 4.4 Mbp, M. tuberculosis is one of the largest known bacterial genomes, coming in just short of E. coli, and a distant third to Streptomyces coelicolor.

The genome of Mycobacterium leprae is 3,268,203 base pairs long, with only 1,604 predicted protein-coding regions, and a G+C content of about 57.8%. Only 49.5% of the M. leprae genome contains open reading frames (protein-coding regions), the rest of the genome is comprised of pseudogenes, which are inactive reading frames with recognizable and functional counterparts in M. tuberculosis (27%), and regions that do not appear to be coding at all, and may be gene remnants mutated beyond recognition (23.5%). Of the genome of M. tuberculosis, 90.8% of the genome contains protein-coding sequences with only 6 pseudogenes, compared to the 1,116 pseudogenes on the M. leprae genome.

iv. Pseudomonas

The genus Pseudomonas is characterized by Gram-negative rods that utilize glucose oxidatively. Members are classified into five groups based on ribosomal RNA homology. These bacteria are resistant to most antibiotics and are capable of surviving in very harsh conditions tolerated by very few other organisms. They also are known to produce a coating that helps protect the bacterium from outside agents. Pseudomonas is often found in hospitals and clinics and, not surprisingly, is a major cause of nosocomal infections. It often targets immunocompromised individuals, such as burn victims and individuals on respirators or with indwelling catheters. Infection sites are varied and include the urinary tract, blood, lungs, and pharynx. However, because it is non-invasive, it tends not to be found in healthy individuals.

Pseudomonas aeruginosa is the most common member of its genus, distinguished from other species of Pseudomonas by its ability to grow at 42° C., produce bluish (pyocyanin) and greenish pigments, and exhibit a characteristic fruity odor. The pathogenicity involves several toxins and chemicals that the bacterium secretes upon infection. The presence of a lipopolysaccharide layer serves to protect the organism as well as aid in cell adherence to host tissues. Lipases and exotoxins secreted by the organism then procede to destroy host cell tissue, leading to complications often associated with infection. P. aeruginosa prefers moist environments, and will grow on almost any laboratory medium. Pseudomonas infections are usually treated with a combination of antibiotics, e.g., an anti-pseudomonal penicillin and an aminoglycoside.

v. Other Bacteria

In addition to the bacteria discussed above, the inventors disclose methods for drug screening, methods for increasing bacterial sensitivity to antibiotics, and methods of reducing bacterial virulence for a variety of other bacteria. Such bacteria include Streptococcus pneumoniae, Streptococcus pyogenes, Streptococcus agalactiae, Streptococcus viridans, Enterococcus faecalis, Enterococcus faecium, Clostridium botulinum, Clostridium perfringens, Clostridium tetani, Clostridium difficile, Listeria monocytogenes, Legionella pneumophila, Francisella tularensis, Pasteurella multocida, Brucella abortive, Brucella suis, Brucella melitensis, Bordetella pertussis, Salmonella sp., Shigella sp., Eschericia coli, Vibrio sp., Klebsiella sp., Aeromonas sp., Plesiomonas sp., Rickettsiae sp., Chlamydiae sp., Ehrlichia sp., Mycoplasma sp., Helicobacter sp., Campylobacter sp., and Haemophilus sp.

B. Fungal Infections

Fungi include yeasts and molds. Examples of fungal infections that may be treated with halides include without limitation Aspergillus spp. including Aspergillus fumigatus, Blastomyces dermatitidis, Candida spp., including Candida albicans, Coccidioides immitis, Cryptococcus neoformans, Histoplasma capsulatum, Pneumocystis carinii, Rhizomucor spp., and Rhizopus spp.

C. Viral Infections

The present invention will be useful in treating a wide variety of viral infections, including those caused by Togoviridae, Flaviviridae, Coronoviridae, Rhabdoviridae, Filoviridae, Paramyxoviridae, Orthomyxoviridae, Bunyaviridae, Arenaviridae, Retroviridae, Herpesviridae, Poxviridae and Iridoviridae. Specific viruses include the human viruses HIV, HSV-1, HSV-2, EBV, CMV, herpesvirus B, HHV6, varicella zoster virus, HHV8, respiratory syncytial virus (RSV), influenza A, B and C viruses, hepatitis A, hepatitis B, hepatitis C, hepatitis G, smallpox, vaccinia virus, Marburg virus, ebola virus, dengue virus, West Nile virus, hantavirus, measles virus, mumps virus, rubella virus, rabies virus, yellow fever virus, Japanese encephalitis virus, Murray Valley encephalitis virus, Rocio virus, tick-borne encephalitis virus, St. Louis encephalitis virus, chikungynya virus, o'nyong-nyong virus, Ross River virus, Mayaro virus, human coronaviruses 229-E and OC43, vesicular stomatitis virus, sandfly fever virus, Rift Valley River virus, Lasa virus, lymphocytic choriomeningitis virus, Machupo virus, Junin virus, HTLV-I and -II. Other animal viruses include those of swine (swinepox, African swine fever virus, hemagluttinating virus of swine, hog cholera virus, pseudorabies virus), sheep (border disease virus, Maedi virus, visna virus), cattle (bovine leukemia virus, bovine diarrhea virus, bovine lentivirus, infectious bovine rhinotracheitis virus), horses (eastern and western equine encephalitis virus, Venezuelan equine encephalitis virus, equine infectious anemia virus, equine arteritis virus), cats (feline immunodeficiency virus, feline leukemia virus, feline infectious peritonitis virus), monkeys (simian hemorrhagic fever virus) and fowl (Marek's disease virus, turkey bluecomb virus, infectious bronchitis virus of fowl, avian reticuloendotheliosis, sarcoma, and leukemia viruses).

D. Specific Pathologic Conditions

i. Human Diseases

The present invention will have particular application to disease states involving infections of mucosal surfaces, such as those of the respiratory tract. One specific example is the treatment of cystic fibrosis patients who are at considerable risk of bacterial and fungal infection of the upper respiratory system. Lung disease in these patients results from clogging of airways due to inflammation. Inflammation and infection cause injury to the lungs and structural changes that lead to a variety of symptoms. In the early stages, incessant coughing, copious phlegm production, and decreased ability to exercise are common. Many of these symptoms occur when bacteria that normally inhabit the thick mucus grow out of control and cause pneumonia. In later stages of CF, changes in the architecture of the lung further exacerbate chronic difficulties in breathing. Aspergillus fumigatus is a common fungus and can also lead to worsening lung disease in people with CF. Another is infection with mycobacterium avium complex (MAC), a group of bacteria related to tuberculosis, which can cause further lung damage and does not respond to common antibiotics.

The lungs of individuals with cystic fibrosis are colonized and infected by bacteria from an early age. These bacteria, which often spread amongst individuals with CF, thrive in the altered mucus, which collects in the small airways of the lungs. This mucus encourages the development of bacterial microenvironments (biofilms) that are difficult for immune cells (and antibiotics) to penetrate. The lungs respond to repeated damage by thick secretions and chronic infections by gradually remodeling the lower airways (bronchiectasis), making infection even more difficult to eradicate. Over time, however, both the types of bacteria and their individual characteristics change in individuals with CF. In the initial stage, common bacteria such as Staphylococcus aureus and Hemophilus influenzae colonize and infect the lungs. Eventually, however, Pseudomonas aeruginosa (and sometimes Burkholderia cepacia) dominates. Once within the lungs, these bacteria adapt to the environment and develop resistance to commonly used antibiotics. Pseudomonas can develop special characteristics that allow the formation of large colonies, known as “mucoid” Pseudomonas and rarely seen in people that do not have CF.

Respiratory viral infections cause an enormous disease burden in infants, children and adults. In persons with underlying cardiopulmonary disease conditions the clinical impact of such common infections is even greater. Some common classes of human disease associated respiratory viruses include: paramyxoviruses, orthomyxoviruses, coronaviruses, adenoviruses, picornavirus, parvoviruses, arenaviruses, herpesviruses, and retroviruses.

Influenza A virus is a common respiratory pathogen that typically infects 10-20% of the population each winter within the U.S. resulting in ˜20,000 deaths and 114,000 hospitalizations (LaForce et al., 1994). Importantly, influenza can also undergo substantial changes (through recombination/antigenic shift) that can leave us with little to no protective immunity and increase influenza's mortality rate, even among healthy young adults. These events, such as the influenza pandemics of 1918 (20-40 million deaths world wide), 1957 (70,000 US deaths), and 1968-69 (34,000 US deaths) and the recent appearance of the H5N1 Avian “bird” influenzas in Asia, further demonstrate that influenza is a significant global public health and bioterrorism concern (Horimoto et al., 2005; Palese, 2004; Fauci, 2005). New approaches are need to protect and treat influenza virus infection. The inventors further propose that the hypohalide-generating system will be effective against other respiratory viral pathogens as well.

ii. Veterinary Applications

Respiratory pathogens cause major disease burdens in animals. One example of a respiratory disease in the commercial industry is “shipping fever” in animals such as cattle and swine. This is a predominantly bacterial disease, although viruses may contribute. The stress associated with animal handling, trucking, and feedlot conditions contribute to the disease pathogenesis. The organisms Mannheimia haemolytica and, less commonly, Pasteurella multocida or Histophilus somni are the major pathogens (refs below). The resultant disease consequences have major financial implications for the industry.

The methods described herein may have other applications in the prevention or treatment of respiratory diseases of mammals and birds. For example, birds such as ducks and geese may carry and transmit avian influenza (H5N1) to humans with potential fatal outcomes. Halide or pseudohalide supplementation in these animals could reduce the potential for zoonotic disease (Czuprynski et al., 2004; Ackermann & Brogden, 2000).

E. Combination Therapies

It is further contemplated that the halide compositions of the present invention may be used in combination with or to enhance the activity of other antimicrobial agents. Combinations with other agents may be useful to allow such other agents to be used at lower doses, thereby reducing concerns over toxicity, or to enhance the activity of such agents whose efficacy has been reduced by microbial resistance, or to effectuate a synergism between the multiple agents such that the combination is more effective than the sum of the efficacy of either agent considered independently. The combination may inhibit microbe replication, reduce symptoms, shorten the duration of infection, and/or reduce microbe burden in the patient.

To effect treatment, one will administer the halide and the “other” therapy in a combined amount effective to kill or inhibit proliferation of the cell. This process may involve administering both agents/therapies at the same time. This may be achieved by administering a single composition or pharmacological formulation that includes both agents, or by administering two distinct compositions or formulations at the same time, where each composition contains one agent.

Alternatively, the halide treatment may precede or follow the other treatment by intervals ranging from minutes to weeks. In embodiments where the two treatments are applied separately, one would generally ensure that a significant period of time did not expire between the time of each delivery, such that the therapies would still be able to exert an advantageously combined effect. In such instances, it is contemplated that one would administer both modalities within about 12-24 hours of each other and, more preferably, within about 6-12 hours of each other, with a delay time of only about 12 hours being most preferred. In some situations, it may be desirable to extend the time period for treatment significantly, however, where several days (2, 3, 4, 5, 6 or 7) to several weeks (1, 2, 3, 4, 5, 6, 7 or 8) lapse between the respective administrations.

It also is conceivable that more than one administration of either agent will be desired. Various combinations may be employed, where the halide is “A” and the other agent/therapy is “B,” as exemplified below:

A/B/A B/A/B B/B/A A/A/B B/A/A A/B/B B/B/B/A B/B/A/B
A/A/B/B A/B/A/B A/B/B/A B/B/A/A B/A/B/A B/A/A/B B/B/B/A
A/A/A/B B/A/A/A A/B/A/A A/A/B/A A/B/B/B B/A/B/B B/B/A/B

Other combinations are contemplated. Antimicrobials which may be combined with the halides of the present invention include, but are not limited to, agents listed below.

i. Antibiotics

Any of a wide variety of antibiotics may be used in combination with the halide-containing compositions of the present invention. The combinations will be selected based on the type of organism to be treated, the severity of the infection, and the overall health of the subject.

TABLE 1
ANTIBIOTICS OF CHOICE FOR COMMON PATHOGENS

| | | |
|---|---|---|
| Pathogen | Antibiotic of First Choice^{a} | Alternative Agents^{a} |

Gram-positive cocci

| | | |
|---|---|---|
| Staphylococcus | | |
| aureus or | | |
| S. epidermidis | | |
| Non- | Penicillin | A first-generation cephalosporin, |
| penicillinase- | | vancomycin, imipenem, or |
| producing | | clindamycin; a fluoroquinolone^{b} |
| Penicillinase- | Penicillinase-resistant | A first-generation cephalosporin, |
| producing | penicillin (e.g., | vancomycin, clindamycin, |
| | oxacillin or nafcillin) | imipenem, |
| Methicillin- | Vancomycin with or | amoxicillin-clavulanic acid, |
| resistant | without | ticarcillin-clavulanic acid, |
| | gentamicin and/or | ampicillin-sulbactam; a |
| | rifampin | fluoroquinolone^{b} |
| | | TMP-SMZ, minocycline |
| Streptococci | | |
| Group A, C, G | Penicillin | A cephalosporin^{a}, vancomycin, |
| | | erythromycin; clarithromycin; |
| | | azithromycin; clindamycin |
| Group B | Penicillin (or ampicillin) | A cephalosporin^{a}, vancomycin, or |
| | | erythromycin |
| Enterococcus | | |
| Endocarditis or | Penicillin (or ampicillin) | Vancomycin with gentamicin |
| other serious | with gentamicin | |
| infection | | |
| Uncomplicated | Ampicillin or amoxicillin | A fluoroquinolone, nitrofurantoin |
| urinary tract | | |
| infection | | |
| Viridans group | Penicillin G (with or | A cephalosporin^{a}, vancomycin |
| | without gentamicin) | |
| S. bovis | Penicillin G | A cephalosporin^{a}, vancomycin |
| S. pneumoniae | Penicillin G | A cephalosporin^{a}, erythromycin, |
| | | chloramphenicol, vancomycin |

Gram-negative cocci

| | | |
|---|---|---|
| Neisseria | Ceftriaxone | Spectinomycin, a fluoroquinolone, |
| gonorrhoeae | | cefoxitin, cefixime, cefotaxime |
| | | (see Appendix E) |
| N. meningitidis | Penicillin G | Third-generation cephalosporin, |
| | | chloramphenicol |
| Moraxella | | |
| (Branhamella) | TMP-SMZ | Amoxicillin-clavulanic acid; an |
| catarrhalis | | erythromycin; clarithromycin |
| | | azithromycin, cefuroxime, |
| | | cefixime, |
| | | third-generation cephalosporin, |
| | | tetracycline |

Gram-positive bacilli

| | | |
|---|---|---|
| Clostridium | Penicillin G | Chloramphenicol, metronidazole, |
| perfringens | | or |
| (and | | clindamycin |
| Clostridium sp.) | | |
| Listeria | Ampicillin with or | TMP-SMZ |
| monocytogenes | without gentamicin | |

Gram-negative bacilli

| | | |
|---|---|---|
| Acinetobacter | Imipenem | Tobramycin, gentamicin, or |
| | | amikacin, |
| | | usually with ticarcillin or |
| | | piperacillin (or similar agent); |
| | | TMP-SMZ |
| Aeromonas | TMP-SMZ | Gentamicin, tobramycin; |
| hydrophila | | imipenem; a |
| | | fluoroquinolone |
| Bacteroides | | |
| Bacteroides sp. | Penicillin G | Clindamycin, cefoxitin, |
| (oropharyngeal) | | metronidazole, chloramphenicol, |
| | | cefotetan, ampicillin-sulbactam |
| B. fragilis | Metronidazole | Clindamycin; ampicillin- |
| strains | | sulbactam; |
| (gastrointestinal | | imipenem; cefoxitin^{c}; cefotetan^{c}; |
| strains) | | ticarcillin-clavulanic acid; |
| | | piperacillin^{c}; chloramphenicol; |
| | | cefmetazole^{c} |
| Campylobacter | A fluoroquinolone (adults) | A tetracycline, gentamicin |
| fetus, | or an erythromycin | |
| jejuni | | |
| Enterobacter sp. | Imipenem | An aminoglycoside and |
| | | piperacillin or |
| | | ticarcillin or mezlocillin; a |
| | | third-generation cephalosporin^{d}; |
| | | TMP-SMZ; aztreonam; a |
| | | fluoroquinolone |
| Escherichia coli | | |
| Uncomplicated | TMP-SMZ | A cephalosporin or a |
| urinary | | fluoroquinolone |
| tract infection | | |
| Recurrent or | A cephalosporin^{e} | Ampicillin with or without an |
| systemic | | aminoglycoside, TMP-SMZ, oral |
| infection | | fluoroquinolones useful in |
| | | recurrent infections, ampicillin- |
| | | sulbactam, ticarcillin-clavulanic |
| | | acid, aztreonam |
| Haemophilus | | |
| influenzae | | |
| (coccobacillary) | Cefotaxime or ceftriaxone | Chloramphenicol; cefuroxime for |
| Life-threatening | | pneumonia) |
| infections | | |
| Upper | TMP-SMZ | Ampicillin or amoxicillin; |
| respiratory | | cefuroxime; a sulfonamide with |
| infections and | | or |
| bronchitis | | without an erythromycin; |
| | | cefuroxime-axetil; third- |
| | | generation |
| | | cephalosporin, amoxicillin- |
| | | clavulanic acid, cefaclor, |
| | | tetracycline; clarithromycin; |
| | | azithromycin |
| Klebsiella | A cephalosporin^{e} | An aminoglycoside, imipenem, |
| pneumoniae | | TMP-SMZ, |
| | | ticarcillin-clavulanic acid, |
| | | ampicillin-sulbactam, aztreonam, |
| | | a |
| | | fluoroquinolone; amoxicillin- |
| | | clavulanic acid |
| Legionella spp. | Erythromycin with rifampin | TMP-SMZ; clarithromycin; |
| | | azithromycin; ciprofloxacin |
| Pasteurella | Penicillin G | Tetracycline, cefuroxime, |
| multocida | | amoxicillin-clavulanic acid, |
| | | ampicillin-sulbactam |
| Proteus sp. | Cefotaxime, ceftizoxime, or | An aminoglycoside; ticarcillin or |
| | ceftriaxone^{f} | piperacillin or mezlocillin; TMP- |
| | | SMZ; amoxicillin-clavulanic |
| | | acid; |
| | | ticarcillin-clavulanic acid, |
| | | ampicillin-sulbactam; a |
| | | fluoroquinolone; aztreonam; |
| | | imipenem |
| Providencia | Cefotaxime, ceftizoxime, or | Imipenem; an aminoglycoside |
| stuartii | ceftriaxone^{f} | often |
| | | combined with ticarcillin or |
| | | piperacillin or similar agent; |
| | | ticarcillin-clavulanic acid; TMP- |
| | | SMZ, a fluoroquinolone; |
| | | aztreonam |
| Pseudomonas | | |
| aeruginosa | | |
| (nonurinary tract | Gentamicin or tobramycin or | An aminoglycoside and |
| infection) | amikacin (combined with | ceftazidime; |
| | ticarcillin, | imipenem, or aztreonam plus an |
| | piperacillin, | aminoglycoside; ciprofloxacin |
| | etc. for serious | |
| | infections) | |
| (urinary tract | Ciprofloxacin | Carbenicillin; ticarcillin, |
| infections) | | piperacillin, or mezlocillin; |
| | | ceftazidime; imipenem; |
| | | aztreonam; |
| | | an aminoglycoside |
| Pseudomonas | TMP-SMZ | Ceftazidime, chloramphenicol |
| cepacia | | |
| Salmonella typhi | Ceftriaxone | Ampicillin, amoxicillin, TMP- |
| | | SMZ, |
| | | chloramphenicol; a |
| | | fluoroquinolone |
| Other species | Cefotaxime or ceftriaxone | Ampicillin or amoxicillin, TMP- |
| | | SMZ, |
| | | chloramphenicol; a |
| | | fluoroquinolone |
| Serratia | Cefotaxime, ceftizoxime, or | Gentamicin or amikacin; |
| | ceftriaxone^{f} | imipenem; |
| | | TMP-SMZ; ticarcillin, |
| | | piperacillin, |
| | | or mezlocillin; aztreonam; a |
| | | fluoroquinolone |
| Shigella | A fluoroquinolone | TMP-SMZ; ceftriaxone; |
| | | ampicillin |
| Vibrio cholerae | A tetracycline | TMP-SMZ; a fluoroquinolone |
| (chlorea) | | |
| Vibrio vulnificus | A tetracycline | Cefotaxime |
| Xanthomonas | TMP-SMZ | Minocycline, ceftazidime, a |
| (Pseudomonas) | | fluoroquinolone |
| maltophilia | | |
| Yersinia | TMP-SMZ | A fluoroquinolone; an |
| enterocolitica | | aminoglycoside; |
| | | cefotaxime or ceftizoxime |
| Yersinia pestis | Streptomycin | A tetracycline; chloramphenicol; |
| (plague) | | gentamicin |

Key: TMP-SMZ = trimethoprim-sulfamethoxazole.
^{a}Choice presumes susceptibility studies indicate that the pathogen is susceptible to the agent.
^{b}The experience with fluoroquinolone use in staphylococcal infections is relatively limited.
The fluoroquinolones should be used only in adults.
^{c}Up to 15-20% of strains may be resistant.
^{d}Enterobacter spp. may develop resistance to the cephalosporins.
^{e}Specific choice will depend on susceptibility studies. Third-generation cephalosporins may be exquisitely active against many Gram-negative bacilli (e.g., E. coli, Klebsiella sp.). In some geographic areas, 20-25% of community-acquired E. coli infections may be resistant to ampicillin (amoxicillin).
^{f}In severely ill patients, this is often combined with an aminoglycoside while awaiting susceptibility data.

TABLE 2
COMMON ANTIBIOTICS AND USUAL ORAL DOSES

| ANTIBIOTIC | DOSAGE |
|---|---|
| Penicillin V | 250 mg qid |
| Rugby (generic) | |
| V-cillin K | |
| Dicloxacillin | 250 mg qid |
| Glenlawn (generic) | |
| Dynapen | |
| Cloxacillin (Tegopen) | 250 mg qid |
| Amoxicillin | 250 mg tid |
| Rugby (generic) | |
| Polymox | |
| Ampicillin | 250 mg qid |
| Moore (generic) | |
| Polycillin | |
| Augmentin | tid |
| 250-mg tablets | |
| chewables (250 mg) | |
| 125-mg (suspension) | |
| chewables (125 mg) | |
| Carbenicillin (Geocillin) | 382 mg qid (1 tb) |
| | 2 tab qid |
| Cephalexin | 250 mg qid |
| Rugby (generic) | |
| Keflex | |
| Rugby (generic) | 500 mg qid |
| Keflex | |
| Cefadroxil | 1 gm bid |
| Rugby (generic) | |
| Duricef | |
| Cephradine | 250 mg qid |
| Rugby (generic | |
| Velosef | |
| Rugby (generic) | 500 mg qid |
| Velosef | |
| Cefaclor | 250 mg tid |
| Ceclor | |
| Cefuroxime axetil | |
| Ceftin | 125 mg bid |
| | 250 mg bid |
| | 500 mg bid |
| Cefixime | 400 mg q24h |
| Suprax | |
| Cefprozil | |
| Cefzil | 250 mg q12h |
| Loracarbef (Lorabid) | 200 mg bid |
| Cefpodoxime proxetil | 200 mg bid |
| (Vantin) | |
| Clindamycin | 300 mg q8h |
| Cleocin | |
| TMP/SMZ | 1 double-strength bid |
| Bactrim | |
| Septra | |
| (generic) | |
| Trimethoprim | 100 mg bid |
| Rugby (generic) | |
| Proloprim | |
| Erythromycin (base) | 250 mg qid |
| Abbott | |
| E-mycin (delayed release) | |
| Erythromycin stearate | 250 mg qid |
| Rugby (generic) | |
| Azithromycin | 1 g once only 500 mg, |
| Zithromax | day 1, plus 250 mg, day |
| | 2-5 |
| Clarithromycin | 250 mg bid |
| Biaxin | 500 mg bid |
| Tetracycline hydrochloride | 250 mg qid |
| Mylan | |
| Sumycin 250 | |
| Doxycycline | 100 mg qd (with 200- |
| Lederle (generic) | mg initial load) |
| Vibramycin | |
| Vancomycin | |
| Vancocin HCl (oral | Capsules |
| soln/powder) | 125 mg q6h PO |
| Metronidazole | 250 mg qid |
| Rugby (generic) | |
| Flagyl | |
| Norfloxacin | 400 mg bid |
| Noroxin | |
| Ciprofloxacin | 250 mg bid |
| Cipro | 500 mg bid |
| | 750 mg bid |
| Ofloxacin | |
| Floxin | 200 mg bid |
| | 300 mg bid |
| | 400 mg bid |
| Lomefloxacin Maxaquin | 400 mg once qd |

TABLE 3
COMMON ANTIBIOTICS AND
USUAL PARENTERAL DOSES

| ANTIBIOTIC | DOSAGE |
|---|---|
| Penicillin G | 2,400,000 units |
| Pfizerpen G (Pfizer) | 12 million units |
| Oxacillin | 12 g |
| Prostaphlin (Bristol) | |
| Nafcillin | 12 g |
| Nafcil (Bristol) | |
| Ampicillin | 6 g |
| Omnipen (Wyeth) | |
| Ticarcillin | 18 g |
| Ticar (Beecham) | |
| Piperacillin | 18 g |
| Pipracil (Lederle) | 16 g |
| Mezlocillin | 18 g |
| Mezlin (Miles) | 16 g |
| Ticarcillin-clavulanate | 18 g/0.6 g |
| Timentin (Beecham) | 12 g/0.4 g |
| Ampicillin-sulbactam | 6 g |
| Unasyn (Roerig) | 12 g |
| Cephalothin | 9 g (1.5 g q4h) |
| Keflin (Lilly) | |
| Cefazolin | 4 g (1 g q6h) |
| Ancef (SKF) | 3 g (1 g q8h) |
| Cefuroxime | 6 g 2.25 g (750 mg q8h) |
| Zinacef (Glaxo) | 4.5 g (1.5 g q8h) |
| Cefamandole | 9 g (1.5 g q4h) |
| Mandol (Lilly) | |
| Cefoxitin | 8 g (2 g q6h) |
| Mefoxin (MSD) | 6 g (2 g q8h) |
| Cefonicid | 1 g q12h |
| Monicid (SKF) | |
| Cefotetan | 2 g q12h |
| Cefotan (Stuart) | |
| Cefmetazole | 2 g q8h |
| Zefazone (Upjohn) | |
| Ceftriaxone | 2 g (2.0 g q24h) |
| Rocephin (Roche) | 1 g (1.0 g q24h) |
| Ceftazidime | 6 g (2 g q8h) |
| Fortax (Glaxo) | |
| Taxicef (SKF) | |
| Tozidime (Lilly) | |
| Cefotaxime | 2 g q6h |
| Claforan (Hoechst) | 2 g q8h |
| Cefoperazone | 8 g (2 g q6h) |
| Cefobid (Pfizer) | 6 g (2 g q8h) |
| Ceftizoxime | (2 g q8h) |
| Ceftizox (SKF) | |
| Aztreonam | 2 g q8h |
| Azactam (Squibb) | 1 g q8h |
| Imipenem | 2000 mg (500 mg 16 h) |
| Primaxin (MSD) | |
| Gentamicin | |
| Garamycin | 360 mg (1.5 mg/kg q8h |
| (Schering) | for an 80-kg patient) |
| (generic) (Elkins-Sinn) | |
| Tobramycin | 360 mg (1.5 mg/kg q8h |
| Nebcin (Dista) | for an 80-kg patient) |
| Amikacin | 1200 mg (7.5 mg/kg |
| Amikin (Bristol) | q12h for an 80-kg |
| | patient) |
| Clindamycin | 2400 mg (600 mg q6h) |
| Cleocin (Upjohn) | 2700 mg (900 mg q8h) |
| | 1800 mg (600 mg q8h) |
| Chloramphenicol | 4 g (1 g q6h) |
| Chloromycetin (P/D) | |
| TMP/SMZ | 1400 mg TMP (5 mg |
| Septra (Burroughs | TMP/kg q6h for a 70-kg |
| Wellcom) | patient) |
| | 700 mg TMP (5 mg |
| | TMP/kg q12h for a 70- |
| | kg patient) |
| Erythromycin | 2000 mg (500 mg q6h) |
| Erythromycin | |
| (Elkins-Sinn) | |
| Doxycycline | 200 mg (100 mg q12h) |
| Vibramycin (Pfizer) | |
| Vancomycin | 2000 mg (500 mg q6h) |
| Vancocin (Lilly) | |
| Metronidazole | 2000 mg (500 mg q6h) |
| (generic) (Elkins-Sinn) | |
| Ciprofloxacin | 200 mg q12h |
| Cipro | 400 mg q12h |
| Pentamidine | 280 mg (4 mg/kg q24h |
| Pentam (LyphoMed) | for a 70-kg patient) |

ii. Antivirals

The present invention also contemplates the use of traditional antiviral therapies in combination with the halide treatments of the present invention. The following discussion provides examples of antiviral therapies that can be combined with halides and halide salts in the treatment of viral infections.

Before cell entry. One anti-viral strategy is to interfere with the ability of a virus to infiltrate a target cell. The virus must go through a sequence of steps to do this, beginning with binding to a specific receptor molecule on the surface of the host cell and ending with the virus uncoating inside the cell and releasing its contents. Viruses that have a lipid envelope must also fuse their envelope with the target cell, or with a vesicle that transports them into the cell, before they can uncoat.

This stage of viral replication can be inhibited in two ways: (a) using agents which mimic the virus-associated protein (VAP) and bind to the cellular receptors. This may include VAP anti-idiotypic antibodies, anti-receptor antibodies, and natural ligands of the receptor and anti-receptor antibodies; (b) using agents which mimic the receptor and bind to the VAP. This includes anti-VAP antibodies, receptor anti-idiotypic antibodies, extraneous receptor and synthetic receptor mimics.

This strategy of designing drugs can be very expensive, and since the process of generating anti-idiotypic antibodies is partly trial and error, it can be a relatively slow process until an adequate molecule is produced.

A very early stage of viral infection is viral entry, when the virus attaches to and enters the host cell. A number of “entry-inhibiting” or “entry-blocking” drugs are being developed to fight HIV. HIV most heavily targets the immune-system white blood cells known as helper T cells, and identifies these target cells through T-cell surface receptors designated “CD4” and “CCR5.” Attempts to interfere with the binding of HIV with the CD4 receptor have failed to stop HIV from infecting helper T cells, but research continues on trying to interfere with the binding of HIV to the CCR5 receptor in hopes that it will be more effective.

However, two entry-blockers, amantadine and rimantadine, have been introduced to combat influenza, and researchers are working on entry-inhibiting drugs to combat hepatitis B and C virus. One entry-blocker is pleconaril. Pleconaril works against rhinoviruses, which cause the common cold, by blocking a pocket on the surface of the virus that controls the uncoating process. This pocket is similar in most strains of rhinoviruses and enteroviruses, which can cause diarrhea, meningitis, conjunctivitis, and encephalitis.

During viral synthesis. A second approach is to target the processes that synthesize virus components after a virus invades a cell. One way of doing this is to develop nucleotide or nucleoside analogues that look like the building blocks of RNA or DNA, but deactivate the enzymes that synthesize the RNA or DNA once the analogue is incorporated. The first successful antiviral, acyclovir, is a nucleoside analogue, and is effective against herpesvirus infections. The first antiviral drug to be approved for treating HIV, zidovudine (AZT), is also a nucleoside analogue.

An improved knowledge of the action of reverse transcriptase has led to better nucleoside analogues to treat HIV infections. One of these drugs, lamivudine, has been approved to treat hepatitis B, which uses reverse transcriptase as part of its replication process. Researchers have gone further and developed inhibitors that do not look like nucleosides, but can still block reverse transcriptase. Other targets being considered for HIV antivirals include RNase H, which is a component of reverse transcriptase that splits the synthesized DNA from the original viral RNA; and integrase, which splices the synthesized DNA into the host cell genome.

Once a virus genome becomes operational in a host cell, it then generates messenger RNA (mRNA) molecules that direct the synthesis of viral proteins. Production of mRNA is initiated by proteins known as transcription factors. Several antivirals are now being designed to block attachment of transcription factors to viral DNA.

Genomics has not only helped find targets for many antivirals, it has provided the basis for an entirely new type of drug, based on “antisense” molecules. These are segments of DNA or RNA that are designed as “mirror images” to critical sections of viral genomes, and the binding of these antisense segments to these target sections blocks the operation of those genomes. A phosphorothioate antisense drug named fomivirsen has been introduced, used to treat opportunistic eye infections in AIDS patients caused by cytomegalovirus, and other antisense antivirals are in development. An antisense structural type that has proven especially valuable in research is morpholino antisense. Morpholino oligos have been used to experimentally suppress many viral types.

Yet another antiviral technique inspired by genomics is a set of drugs based on ribozymes, which are enzymes that will cut apart viral RNA or DNA at selected sites. In their natural course, ribozymes are used as part of the viral manufacturing sequence, but these synthetic ribozymes are designed to cut RNA and DNA at sites that will disable them.

A ribozyme antiviral to deal with hepatitis C is in field testing, and ribozyme antivirals are being developed to deal with HIV. An interesting variation of this idea is the use of genetically modified cells that can produce custom-tailored ribozymes. This is part of a broader effort to create genetically modified cells that can be injected into a host to attack pathogens by generating specialized proteins that block viral replication at various phases of the viral life cycle.

Some viruses include an enzyme known as a protease that cuts viral protein chains apart so they can be assembled into their final configuration. HIV includes a protease, and so considerable research has been performed to find “protease inhibitors” to attack HIV at that phase of its life-cycle. Protease inhibitors became available in the 1990's and have proven effective, though they can have unusual side-effects, for example causing fat to build up in unusual places. Improved protease inhibitors are now in development.

Release phase. The final stage in the life cycle of a virus is the release of completed viruses from the host cell, and this step has also been targeted by antiviral drug developers. Two drugs named zanamivir (Relenza) and oseltamivir (Tamiflu) that have been recently introduced to treat influenza prevent the release of viral particles by blocking a molecule named neuraminidase that is found on the surface of flu viruses, and also seems to be constant across a wide range of flu strains.

iii. Anti-Fungal Agents

A variety of anti-fungal agents, for use in combination with halides, are known to those of skill in the art. For example, allylamines and other non-azole ergosterol biosynthesis inhibitors are used and generally act to reduce ergosterol biosynthesis and are thus conceptually related to the azole antifungal agents. Examples include terbinafine, which inhibits squalene epoxidase. Another group of compounds are the antimetabolites, such as flucytosine, which leads to incorrect DNA synthesis. Azoles are one of the most widely used agents; the compounds inhibit the synthesis of ergosterol by blocking the action of 14-α-demethylase. An example is fluconazole. Glucan synthesis inhibitors affect glucan, a key component of the fungal cell wall. One example is caspofungin. Polyenes are potent agents acting by binding to the fungal cell membrane and causing the fungus to leak electrolytes. Amphotericin B falls into this group. Finally, griseofulvin acts by disrupting the mitotic spindle and thus constitutes yet another class of agents.

iv. Peroxidases

Another combination therapy includes the use of peroxidases in conjunction with halides. Specific peroxidases for combination therapies include lactoperoxidase, myeloperoxidase, and horseradish peroxidase. Also relevant in combination therapies are the treatments disclosed in U.S. Pat. No. 6,702,998, U.S. Pat. No. 5,503,853 and EP 0 361 908.

F. Routes and Modes of Administration

The proper dosage of a halide necessary to prevent microbial growth and proliferation depends upon a number of factors including the types of microbe that might be present, the environment into which the halide is being introduced, and the time that the halide is envisioned to remain in a given area.

i. Topical Delivery

In accordance with the present invention, there will be provided various devices and preparations that will assist in topical, transdermal and percutaneous delivery of the avicin/agent compositions described herein. Although devices and formulations that impart their own effects on transport maybe utilized, it is not necessary that the devices or preparations used herein provide any more than a structural role to contain the avicin/agent compositions and to provide a means of bringing such compositions into contact with the appropriate tissue.

In one topical embodiment, the present invention can utilize a patch. A transdermal or “skin” patch is a medicated adhesive patch that is placed on the skin to deliver a time released dose of medication through the skin and into the bloodstream. A wide variety of pharmaceuticals can be delivered by transdermal patches. The first commercially available prescription patch was approved by the U.S. Food and Drug Administration in December 1979, which administered scopolamine for motion sickness.

The main components to a transdermal patch are (a) a liner to protect the patch during storage (removed prior to use); (b) the active agent; (c) an adhesive that serves to adhere the components of the patch together along with adhering the patch to the skin; (d) a membrane to control the release of the drug from the reservoir and multi-layer patches; and (e) a backing that protects the patch from the outer environment.

There are four main types of transdermal patches. Single-layer Drug-in-Adhesive patches have an adhesive layer that also contains the agent. In this type of patch the adhesive layer not only serves to adhere the various layers together, along with the entire system to the skin, but is also responsible for the releasing of the drug. The adhesive layer is surrounded by a temporary liner and a backing. Multi-layer Drug-in-Adhesive patches are similar to the single-layer system in that both adhesive layers are also responsible for the releasing of the drug. The multi-layer system is different however that it adds another layer of drug-in-adhesive, usually separated by a membrane (but not in all cases). This patch also has a temporary liner-layer and a permanent backing. Reservoir patches are unlike the Single-layer and Multi-layer Drug-in-Adhesive systems in that the reservoir transdermal system has a separate drug layer. The drug layer is a liquid compartment containing a drug solution or suspension separated by the adhesive layer. This patch is also backed by the backing layer. In this type of system the rate of release is zero order. Matrix patches have a drug layer of a semisolid matrix containing a drug solution or suspension. The adhesive layer in this patch surrounds the drug layer partially overlaying it.

Various alternate sites of administration will also find use with the subject invention. For instance, the compositions of the invention may be formulated, in addition to the formulations discussed above, in suppositories, douches, aerosol and intranasal compositions. Intranasal formulations may be prepared which include vehicles that neither cause irritation to the nasal mucosa nor significantly disturb ciliary function. Diluents such as water, aqueous saline or other known substances can be employed with the subject invention. The nasal formulations also may contain preservatives such as, but not limited to, chlorobutanol and benzalkonium chloride.

In certain embodiments, a cancer may treated after surgical excision to eliminate microscopic residual disease. Both at the time of surgery, and thereafter (periodically or continuously), the therapeutic compositions of the present invention can be administered to the body cavity. This is, in essence, a topical treatment of the surface of the cavity. The volume of the composition should be sufficient to ensure that the entire surface of the cavity is contacted by the expression construct. By analogy, a post-operative field may be treated to prevent or inhibit infection.

In one embodiment, administration simply will entail injection of the therapeutic composition into the cavity formed by the surgery/tumor excision. In another embodiment, mechanical application via a sponge, swab or other device may be desired. Either of these approaches can be used subsequent to the tumor removal as well as during the initial surgery. In still another embodiment, a catheter is inserted into the cavity prior to closure of the surgical entry site. The cavity may then be continuously perfused for a desired period of time.

In another form of this treatment, the topical application of the therapeutic composition is targeted at a natural body cavity such as the mouth, pharynx, esophagus, larynx, trachea, pleural cavity, peritoneal cavity, or hollow organ cavities including the bladder, colon or other visceral organs. Again, a variety of methods may be employed to affect the topical application into these visceral organs or cavity surfaces. For example, the oral cavity in the pharynx may be affected by simply oral swishing and gargling with solutions.

In another topical delivery embodiment, the present invention will utilize a fluid or semi-fluid vehicle. Non-limiting examples of suitable vehicles include emulsions (e.g., water-in-oil, water-in-oil-in-water, oil-in-water, oil-in-water-in-oil, oil-in-water-in-silicone, water-in-silicone, silicone-in-water emulsions), creams, lotions, solutions (both aqueous and hydro-alcoholic), anhydrous bases (such as lipsticks and powders), gels, powdered and liquid aerosols, ointments, and other combination of the forgoing as would be known to one of ordinary skill in the art (see, e.g., Remington's, 1990 and International Cosmetic Ingredient Dictionary and Handbook, 10^{th} ed., 2004). Variations and other appropriate vehicles will be apparent to the skilled artisan and are appropriate for use in the present invention. In certain aspects, it is important that the concentrations and combinations of the compounds, ingredients, and agents be selected in such a way that the combinations are chemically compatible and do not form complexes which precipitate from the finished product.

ii. Oral Formulations

Administration of certain embodiments of the pharmaceutical compositions set forth herein will be via any common route so long as the target tissue is available via that route. For example, this includes esophageal, gastric, oral, nasal, buccal, anal, rectal, vaginal, topical ophthalmic, or applications to skin. Such compositions would normally be administered as pharmaceutically acceptable compositions that include physiologically acceptable carriers, buffers or other excipients. Examples of other excipients include fragrances and flavorants.

The formulation may be in a liquid form or suspension. A typical composition for such purpose comprises a pharmaceutically acceptable carrier. For instance, the composition may contain 10 mg, 25 mg, 50 mg or up to about 100 mg of human serum albumin per ml of phosphate buffered saline. Other pharmaceutically acceptable carriers include aqueous solutions, non-toxic excipients, including salts, preservatives, buffers and the like. Examples of non-aqueous solvents are propylene glycol, polyethylene glycol, vegetable oil and injectable organic esters such as ethyloleate. Aqueous carriers include water, alcoholic/aqueous solutions, saline solutions, parenteral vehicles such as sodium chloride, Ringer's dextrose, etc. Preservatives include antimicrobial agents, anti-oxidants, chelating agents and inert gases. The pH and exact concentration of the various components of the pharmaceutical composition are adjusted according to well-known parameters.

Examples of aqueous compositions for oral administration include a mouthwash, mouthrinse, a coating for application to the mouth via an applicator, or mouthspray. Mouthwash formulations are well-known to those of skill in the art. Formulations pertaining to mouthwashes and oral rinses are discussed in detail, for example, in U.S. Pat. No. 6,387,352, U.S. Pat. No. 6,348,187, U.S. Pat. No. 6,171,611, U.S. Pat. No. 6,165,494, U.S. Pat. No. 6,117,417, U.S. Pat. No. 5,993,785, U.S. Pat. No. 5,695,746, U.S. Pat. No. 5,470,561, U.S. Pat. No. 4,919,918, U.S. Patent Appn. 20040076590, U.S. Patent Appn. 20030152530, and U.S. Patent Appn. 20020044910, each of which is herein specifically incorporated by reference into this section of the specification and all other sections of the specification.

Oral formulations include such normally employed excipients as, for example, pharmaceutical grades of mannitol, lactose, starch, magnesium stearate, sodium saccharine, cellulose, magnesium carbonate and/or the like. These compositions take the form of solutions such as mouthwashes and mouthrinses. Such compositions and/or preparations should contain at least 0.1% of active compound. The percentage of the compositions and/or preparations may, of course, be varied and/or may conveniently be between about 2 to about 75% of the weight of the unit, and/or preferably between 25-60%. The amount of active compounds in such therapeutically useful compositions is such that a suitable dosage will be obtained.

For oral administration the expression cassette of the present invention may be incorporated with excipients and used in the form of non-ingestible mouthwashes and dentifrices. A mouthwash may be prepared incorporating the active ingredient in the required amount in an appropriate solvent, such as a sodium borate solution (Dobell's Solution). Alternatively, the active ingredient may be incorporated into an antiseptic wash containing sodium borate, glycerin and potassium bicarbonate. The active ingredient also may be dispersed in dentifrices, including: gels, pastes, powders and slurries. The compositions of the present invention may be formulated in a neutral or salt form. Pharmaceutically-acceptable salts include the acid addition salts (formed with the free amino groups of the protein) and which are formed with inorganic acids such as, for example, hydrochloric or phosphoric acids, or such organic acids as acetic, oxalic, tartaric, mandelic, and the like. Salts formed with the free carboxyl groups can also be derived from inorganic bases such as, for example, sodium, potassium, ammonium, calcium, or ferric hydroxides, and such organic bases as isopropylamine, trimethylamine, histidine, procaine and the like.

For oral administration the expression cassette of the present invention may also be incorporated with dyes to aid in the detection of hyperproliferative lesions such as toluidene blue O dye and used in the form of non-digestible mouthwashes, oral renses and dentrifrices. A mouthwash may be prepared incorporating the active ingredient in the required amount in an orally administered dye composition, such as a composition of toluidene blue O dye, a buffer, a flavorant, a preservative, acetic acid, ethyl alcohol and water. Methods and formulations pertaining to the use of Toluidene Blue O dye in the staining of precancerous and cancerous lesions may be found in, for example, U.S. Pat. No. 4,321,251, U.S. Pat. No. 5,372,801, U.S. Pat. No. 6,086,852, and U.S. Patent Appn. 20040146919, each of which is specifically incorporated by reference in its entireity.

Examples of aqueous compositions for application to topical surfaces include emulsions or pharmaceutically acceptable carriers such as solutions of the active compounds as free base or pharmacologically acceptable salts, active compounds mixed with water and a surfactant, and emulsions. Emulsions are typically heterogenous systems of one liquid dispersed in another in the form of droplets usually exceeding 0.1 um in diameter. (Idson, 1988). Emulsions are often biphasic systems comprising of two immiscible liquid phases intimately mixed and dispersed with each other. In general, emulsions may be either water in oil (w/o) or of the oil in water (o/w) variety. Methods pertaining to emulsions that may be used with the methods and compositions of the present invention set forth in U.S. Pat. No. 6,841,539 and U.S. Pat. No. 5,830,499, each of which is herein specifically incorporated by reference in its entirety. Aqueous compositions for application to the skin may also include dispersions in glycerol, liquid polyethylene glycols and mixtures thereof. Under ordinary conditions of storage and use, these preparations contain a preservative to prevent the growth of microorganisms.

The use of liposomes and/or nanoparticles is also contemplated in the present invention. The formation and use of liposomes is generally known to those of skill in the art, and is also described below. Liposomes are also discussed elsewhere in this specification.

Nanocapsules can generally entrap compounds in a stable and reproducible way. To avoid side effects due to intracellular polymeric overloading, such ultrafine particles (sized around 0.1 μm) should be designed using polymers able to be degraded in vivo. Biodegradable polyalkyl-cyanoacrylate nanoparticles that meet these requirements are contemplated for use in the present invention, and such particles may be are easily made. Methods pertaining to the use of nanoparticles that may be used with the methods and compositions of the present invention include U.S. Pat. No. 6,555,376, U.S. Pat. No. 6,797,704, U.S. Patent Appn. 20050143336, U.S. Patent Appn. 20050196343 and U.S. Patent Appn. 20050260276, each of which is herein specifically incorporated by reference in its entireity.

Examples of aqueous compositions contemplated for esophageal or stomach delivery include liquid antacids and liquid alginate-raft forming compositions. Liquid antacids and liquid sucralfate or alginate-raft forming compositions are well known to those skilled in the art. Alginates are pharmaceutical excipients generally regarded as safe and used therefore to prepare a variety of pharmaceutical systems well documented in the patent literature, for example, in U.S. U.S. Pat. No. 6,348,502, U.S. Pat. No. 6,166,084, U.S. Pat. No. 6,166,043, U.S. Pat. No. 6,166,004, U.S. Pat. No. 6,165,615 and U.S. Pat. No. 5,681,827, each of which is herein specifically incorporated by reference into this section of the specification and all other sections of the specification.

Oral formulations contemplated for esophageal or stomach delivery include such normally employed excipients as, for example, pharmaceutical grades of hydroxylethyl cellulose, water, simethicone, sodium carbonate, sodium saccharin, sorbital and/or the like. Flavorants may also be employed. Such compositions and/or preparations should contain at least 0.1% of active compound. The percentage of the compositions and/or preparations may, of course, be varied and/or may conveniently be between about 2 to about 75% of the weight of the unit, and/or preferably between 25-60%. The amount of active compounds in such therapeutically useful compositions is such that a suitable dosage will be obtained.

One may also use solutions and/or sprays, hyposprays, aerosols and/or inhalants in the present invention for administration. One example is a spray for administration to the aerodigestive tract. The sprays are isotonic and/or slightly buffered to maintain a pH of 5.5 to 6.5. In addition, antimicrobial preservatives, similar to those used in ophthalmic preparations, and/or appropriate drug stabilizers, if required, may be included in the formulation. Methods pertaining to spay administration are set forth in U.S. Pat. No. 6,610,272 U.S. Pat. No. 6,551,578 U.S. Pat. No. 6,503,481, U.S. Pat. No. 5,250,298 and U.S. Pat. No. 5,158,761, each of which is specifically incorporated by reference into this section of the specification and all other sections of the specification.

iii. Inhalers

The present invention, in certain embodiments, encompasses the use of aerosol delivery to the respiratory tract. To deliver halides to the respiratory tract, an ideal vehicle is an inhaler device. A large number of inhalers are known in the field and described in the literature. The following U.S. Patents describe inhalers suitable for use according to the present invention:

TABLE 4
INAHLER PATENTS

| | |
|---|---|
| 7,305,986 | Unit dose capsules for use in a dry powder inhaler |
| 7,299,801 | Metering valve for a metered dose inhaler providing |
| | consistent delivery |
| 7,284,553 | Powder inhaler comprising a chamber for a capsule for |
| | taking up a non-returnable capsule being filled with |
| | an active ingredient |
| 7,284,552 | Inhaler device |
| 7,270,124 | Inhaler |
| 7,258,118 | Pharmaceutical powder cartridge, and inhaler equipped |
| | with same |
| 7,252,087 | Powder inhaler |
| 7,234,459 | Nebuliser device for an inhaler apparatus and inhaler |
| | apparatus with such nebuliser device |
| 7,228,860 | Inhaler with vibrational powder dislodgement |
| D544,093 | Inhaler |
| 7,186,958 | Inhaler |
| 7,163,014 | Disposable inhaler system |
| 7,131,441 | Inhaler for multiple dosed administration of a |
| | pharmacological dry powder |
| 7,107,988 | Powder inhaler |
| 7,107,987 | Spacer for delivery of medications from an inhaler to |
| | children and breathing impaired patients |
| 7,090,870 | Dry power inhaler excipient, process for its |
| | preparation and pharmaceutical compositions |
| | containing it |
| 7,077,130 | Disposable inhaler system |
| 7,069,929 | Dry powder inhaler |
| 7,047,967 | Inhaler |
| 6,983,748 | Dry powder inhaler |
| 6,971,384 | Dry powder inhaler |
| 6,971,383 | Dry powder inhaler devices, multi-dose dry powder |
| | drug packages, control systems, and associated |
| | methods |
| 6,955,169 | Inhaler device |
| 6,948,495 | Powder inhaler |
| 6,941,947 | Unit dose dry powder inhaler |
| 6,932,083 | Housing for an inhaler |
| 6,926,003 | Multidose powder inhaler |
| 6,886,560 | Moisture protected powder inhaler |
| 6,880,555 | Inhaler |
| 6,871,647 | Inhaler |
| 6,866,037 | Inhaler |
| 6,860,262 | Inhaler |
| 6,845,772 | Inhaler |
| 6,830,046 | Metered dose inhaler |
| 6,823,863 | Inhaler |
| 6,820,612 | Inhaler holster |
| 6,814,072 | Powder inhaler |
| 6,810,875 | Mouthpiece for a particulate inhaler |
| 6,810,874 | Powder inhaler for combined medicament |
| 6,810,873 | Powder inhaler for combined medicament |
| 6,779,520 | Breath actuated dry powder inhaler |
| 6,769,601 | Inhaler with a dose counter |
| 6,755,190 | Inhaler |
| 6,752,148 | Medicament dry powder inhaler dispensing device |
| 6,745,761 | Inhaler |
| 6,718,972 | Dose metering system for medicament inhaler |
| 6,718,969 | Medication dosage inhaler system |
| 6,715,486 | Dry powder inhaler |
| 6,708,688 | Metered dosage inhaler system with variable positive |
| | pressure settings |
| 6,701,928 | Inhaler dispensing system adapters for |
| | laryngectomized subjects and associated methods |
| 6,701,917 | Dose counter for medicament inhaler |
| 6,698,425 | Powder inhaler |
| 6,698,422 | Canister inhaler having a spacer and easy to operate |
| | lever mechanism and a flexible, elastic mouthpiece |
| 6,684,879 | Inhaler |
| 6,655,381 | Pre-metered dose magazine for breath-actuated dry |
| | powder inhaler |
| 6,651,650 | Ultrasonic atomizer, ultrasonic inhaler and method of |
| | controlling same |
| 6,648,848 | Inhaler for powdered medicaments |
| 6,644,305 | Nasal inhaler |
| 6,629,524 | Inhaler |
| 6,626,173 | Dry powder inhaler |
| 6,622,723 | Inhaler dosing device |
| 6,615,826 | Slow spray metered dose inhaler |
| 6,595,204 | Spacer for an inhaler |
| 6,591,833 | Inhaler apparatus with modified surfaces for enhanced |
| | release of dry powders |
| 6,553,987 | Dry powder inhaler |
| D469,866 | Inhaler for dispensing medication |
| D469,527 | Pharmacological inhaler |
| 6,488,027 | Powder inhaler |
| 6,453,900 | Inhaler device |
| 6,446,627 | Inhaler dose counter |
| 6,427,688 | Dry powder inhaler |
| 6,427,683 | Aerosol inhaler device |
| 6,425,392 | Breath-activated metered-dose inhaler |
| 6,422,236 | Continuous dry powder inhaler |
| 6,415,784 | Inhaler |
| 6,415,526 | Apparatus and method for measuring alignment of |
| | metered dose inhaler valves |
| 6,413,497 | Pharmaceutical composition using a mixture of |
| | propellant gases for a metered dose inhaler |
| 6,405,934 | Optimized liquid droplet spray device for an inhaler |
| | suitable for respiratory therapies |
| 6,405,727 | Inhaler mechanism |
| 6,401,712 | Inhaler |
| 6,397,837 | Inhaler assistive device |
| 6,394,085 | Inhaler spacer |
| 6,390,088 | Aerosol inhaler |
| 6,347,629 | Powder inhaler |
| 6,332,461 | Powder inhaler |
| 6,328,035 | Pneumatic breath actuated inhaler |
| 6,328,034 | Dry powder inhaler |
| 6,328,033 | Powder inhaler |
| 6,325,063 | Breath-powered mist inhaler |
| 6,325,062 | Breath-activated metered-dose inhaler |
| 6,318,361 | Breath-activated metered-dose inhaler |
| 6,305,582 | Inhaler and valve therefor |
| 6,298,847 | Inhaler apparatus with modified surfaces for enhanced |
| | release of dry powders |
| 6,286,507 | Single dose inhaler I |
| 6,285,731 | Counting device and inhaler including a counting |
| | device |
| 6,273,085 | Dry powder inhaler |
| 6,260,549 | Breath-activated metered-dose inhaler |
| 6,253,762 | Metered dose inhaler for fluticasone propionate |
| 6,240,918 | Powdered medication inhaler |
| 6,240,917 | Aerosol holding chamber for a metered-dose inhaler |
| 6,234,169 | Inhaler |
| 6,230,707 | Powder inhaler |
| 6,223,746 | Metered dose inhaler pump |

### IV. Examples

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

### Background

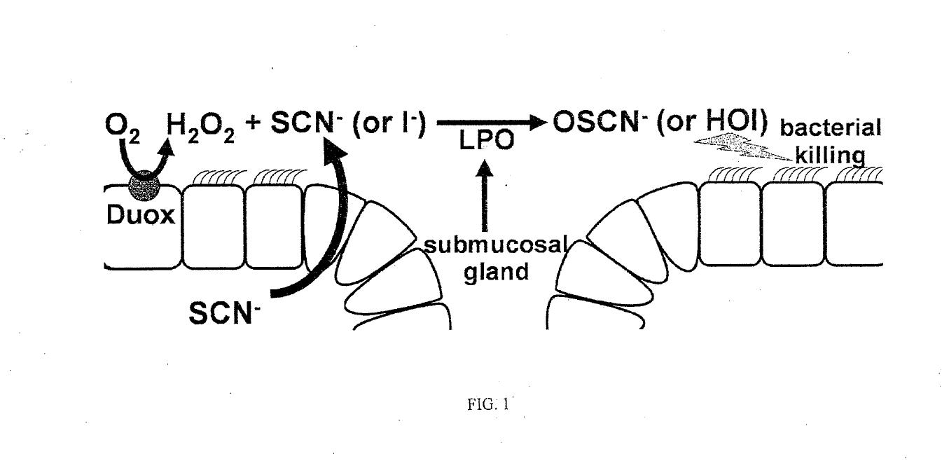

Background. An oxidative host defense system at mucosal surfaces. Recent evidence suggests that airway sterility is preserved not only by mucociliary clearance and antimicrobial polypeptides, but also by an oxidative host defense mechanism. The inventors and others have shown that the oxidative mechanism kills bacteria by producing bactericidal OSCN⁻ in a LPO catalyzed reaction: H₂O₂+SCN⁻→OSCN⁻. OSCN⁻ production requires three processes working in concert: 1) LPO secretion by submucosal glands, 2) H₂O₂ generation by the Duox enzymes of airway epithelia, and 3) SCN⁻ secretion (FIG. 1).

Currently, the mechanism by which OSCN⁻ eliminates bacteria is not known, but OSCN⁻ can oxidize thiol groups in surface proteins thereby mediating conformational change. Importantly, OSCN⁻ is not toxic to eukaryotic cells. LPO has high affinity not only for SCN⁻ but also for I⁻, and LPO can catalyze the oxidation of I⁻ to HOI in the presence of H₂O₂ (FIG. 1). Although I⁻ is not a physiological component of the airway surface fluid, its delivery to the airway lumen could allow HOI generation (in addition to OSCN⁻) by the LPO-Duox enzymes. This could be relevant to infection prevention or treatment because 1) HOI is more efficacious against several bacterial strains than OSCN⁻ and, 2) HOI is also a potent antiviral agent (Belding, 1970). Nevertheless, respiratory viruses (including influenza virus, SARS-coronavirus) have not been tested for their sensitivity to HOI because a Duox-LPO-dependent antimicrobial activity was only recently reported and because I⁻ delivery to airways has not been hitherto proposed.

### Example 2

### Results

Supporting evidence for therapeutic or prophylactic application of the respiratory Duox/LPO system. Mechanisms of SCN⁻ and I⁻ delivery to the mucosal airway surface following their oral or parenteral administration. Published data from the inventors' and other laboratories showed that primary airway epithelia secrete SCN⁻ (Moskwa, 2007) as well as iodide (Fragoso, 2004) if these anions are present at the basolateral side in concentrations higher than 1 μM (I⁻) or 5 μM (SCN⁻). The Cystic Fibrosis Transmembrane Conductance Regulator (CFTR) anion channel and Sodium-Iodide Symporter (NIS) are the main cellular anion transport pathways required for SCN⁻ and I⁻ secretion in vitro (Moskwa, 2007; Conner, 2007; Pedemonte, 2007}. Whereas the in vivo expression of CFTR has been extensively characterized, there has been little evidence hitherto for the in vivo airway expression of NIS.

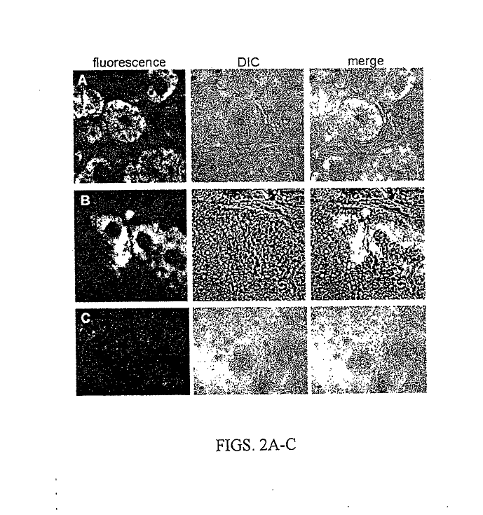

Therefore, the inventors analyzed NIS expression both in nasal brushings (using RT-PCR) and in cross-sections of human trachea (using immunofluorescence). Both RT-PCR (not shown) and immunofluorescence (FIGS. 2A-C) experiments demonstrated NIS expression in the human airway, indicating that I⁻ could be secreted in the airways if the serum concentration of I⁻ was increased either by oral or parenteral administration of I⁻. These data also suggest that secreted SCN⁻ (and potentially I⁻) remains on the apical side of airway epithelial cells, since the rate of apical-to-basolateral leakage is low (<10%/hour) (Moskwa, 2007).

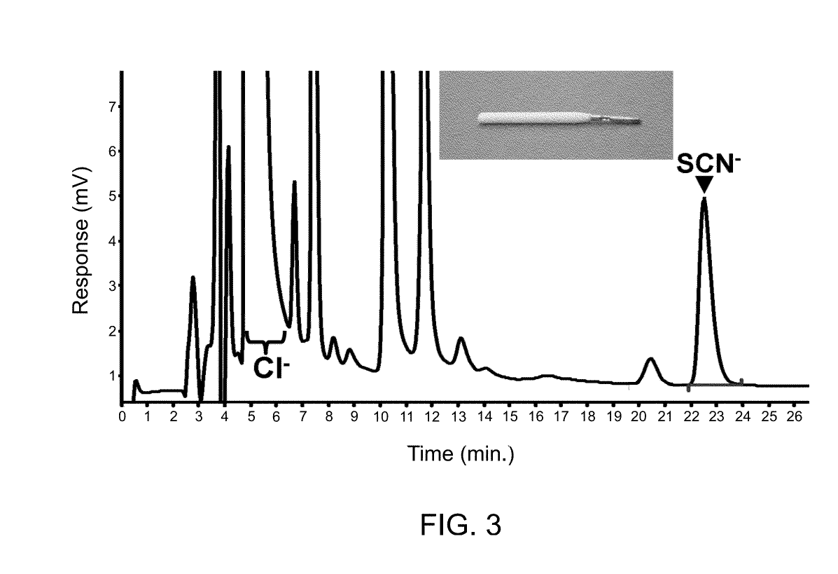

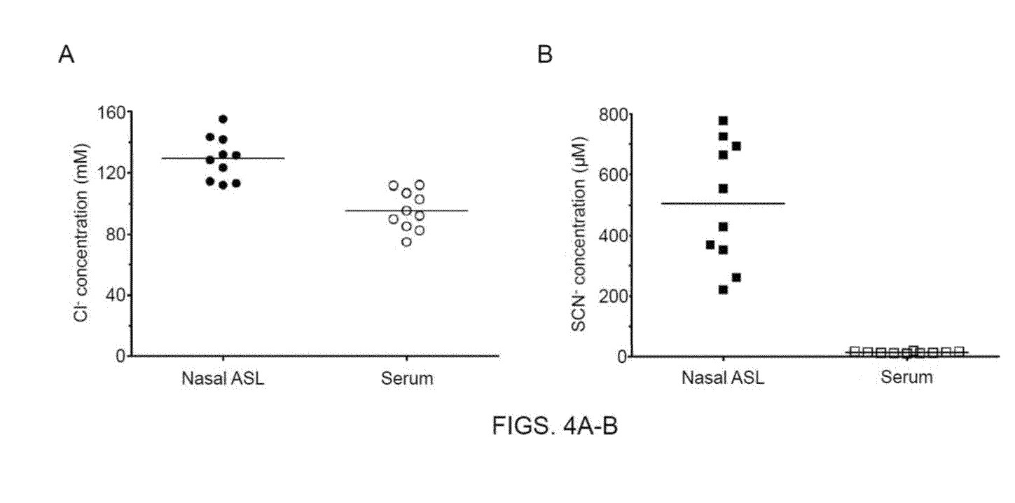

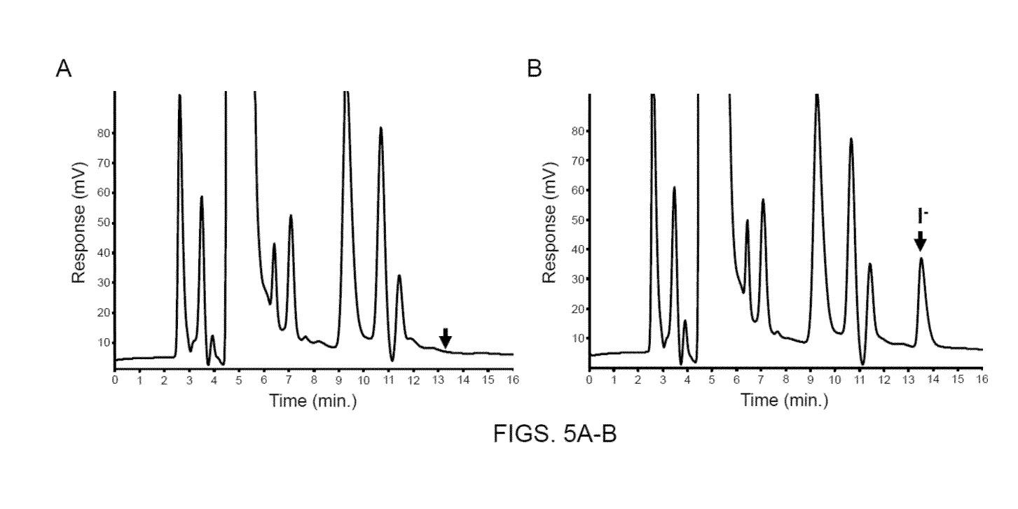

Measurement of airway surface liquid and serum concentrations of SCN⁻, I⁻. The inventors have established a method for the collection of undiluted human nasal airway surface liquid (ASL) and techniques to analyze the anion composition of human ASL and serum. Microcapillary probes developed by Olympus (see inset in FIG. 3) are placed on the inferior turbinates of healthy donors for 15-30 sec. ASL is extracted from the probes by centrifugation. Human serum samples are spun through a series of filters with 100 kDa, 50 kDa, and 3 kDa cut-offs. ASL and filtered serum samples are analyzed for anion composition using ion-exchange chromatography (FIG. 3). These experiments showed that 1) undiluted ASL can be reproducibly collected since the Cl⁻ concentration was in the previously reported range (FIG. 4A), and 2) SCN⁻ concentration in the nasal airway fluid is 250-600 μM (FIG. 4B). By analyzing standard iodide solutions (containing known amounts of I⁻, not shown) and ASL samples, the inventors verified that the ion-exchange method is also suitable to detect iodide in ASL (FIGS. 5A-B). In summary, the inventors established a new approach to collect nasal ASL and to analyze the SCN⁻ and I⁻ concentrations of ASL and serum with ˜0.5 μM lower detection limits.

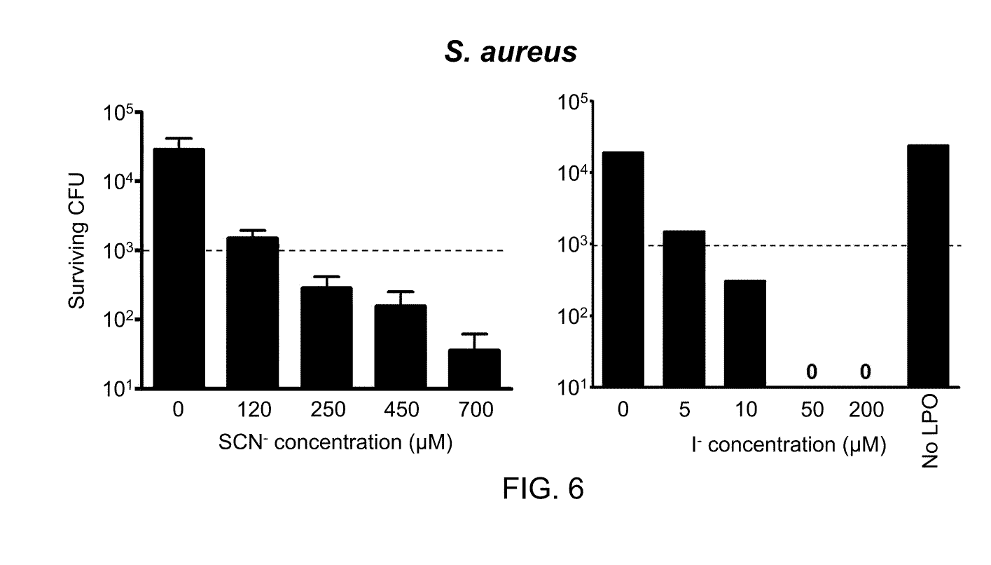

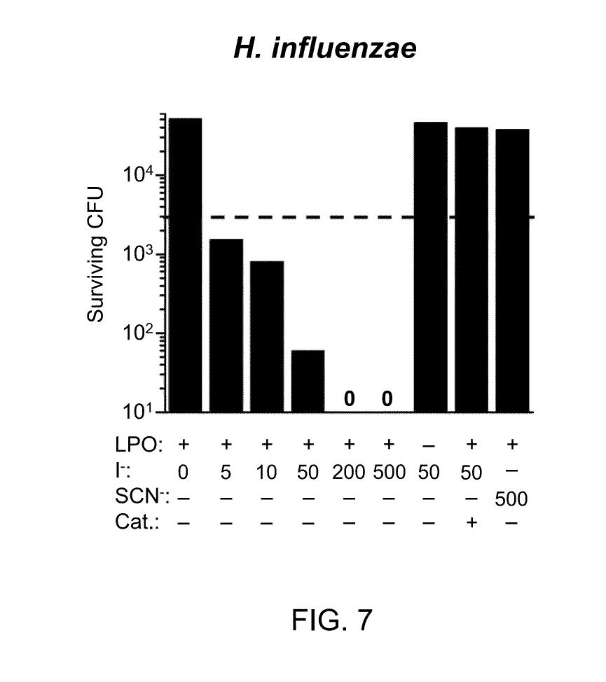

Antibacterial effects of HOI. The inventors tested the bactericidal activity of the oxidative system of airway epithelia in the presence of various I⁻ concentrations (0-500 μM). They used S. aureus and H. influenzae, a Gram-positive and a Gram-negative pathogen, respectively, for bacterial killing assays. Approximately 1000 CFU of each strain were added to the apical chamber of differentiated, non-stimulated airway epithelial cell cultures in 25 μl assay buffer (PBS) together with various combinations of I⁻, LPO, and catalase. In some control samples, the inventors replaced I⁻ with 500 μM SCN⁻. After a 3 hr incubation, the upper chamber liquid was collected, and airway epithelial cells were lysed with saponin to release adherent microbes. These two samples were pooled, and the number of surviving bacteria was determined using quantitative culture. Numbers of surviving S. aureus and H. influenzae were lower after incubating them in the presence of LPO, airway epithelia, and I⁻ if the I⁻ concentration was equal to or greater than 10 μM. Complete eradication of bacteria was detected at 50-200 μM I⁻ (FIGS. 6 and 7). Either catalase or the lack of LPO prevented bacteria killing, indicating the H₂O₂ and LPO-dependence of the antibacterial activity. Importantly, the HOI producing system was more efficient against S. aureus than the OSCN⁻ producing mechanism (FIG. 6). The difference was even greater when H. influenzae was tested: OSCN⁻ was ineffective against this bacterium, whereas HOI demonstrated strong bactericidal activity against H. influenzae (FIG. 7).

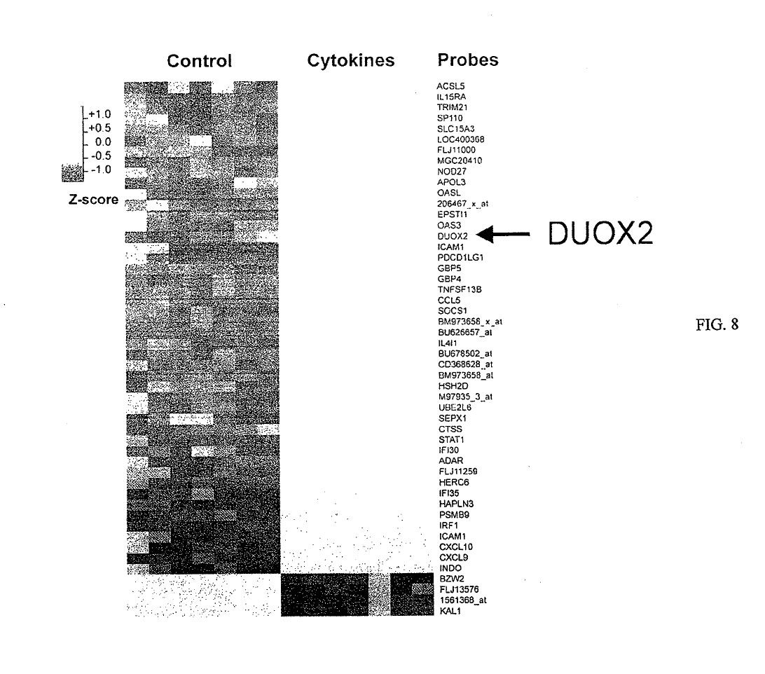

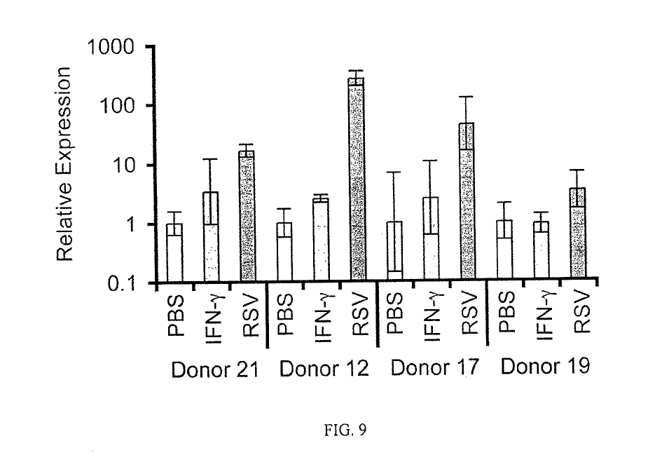

Duox2 is expressed by airway epithelia and expression is regulated in response to viral infection and interferon-γ. The inventors stimulated primary air-liquid interface cultures of human airway epithelia with IL-1β, TNF-α, and interferon-γ (100 ng/ml, n=7) for 24, then performed large scale expression profiling using microarrary hybridization. As shown in FIG. 8, one of the most inducible transcripts in human airway epithelia is Duox2. This observation was confirmed in a subsequent experiment in which human airway epithelia were exposed to interferon-γ or RSV (MOI 4) for 24 hr, followed by RNA extraction and quantitative RT-PCR. FIG. 9 confirms that both IFN-γ and RSV infection increase Duox2 mRNA levels.

Evidence for the inactivation of respiratory viruses by the Duox/LPO system. In a series of studies, the inventors discovered that OSCN⁻ and HOI exhibit differential inhibitory effects against several enveloped and encapsidated RNA and DNA respiratory viruses including RSV, adenovirus, SARS-coronavirus, and influenza.

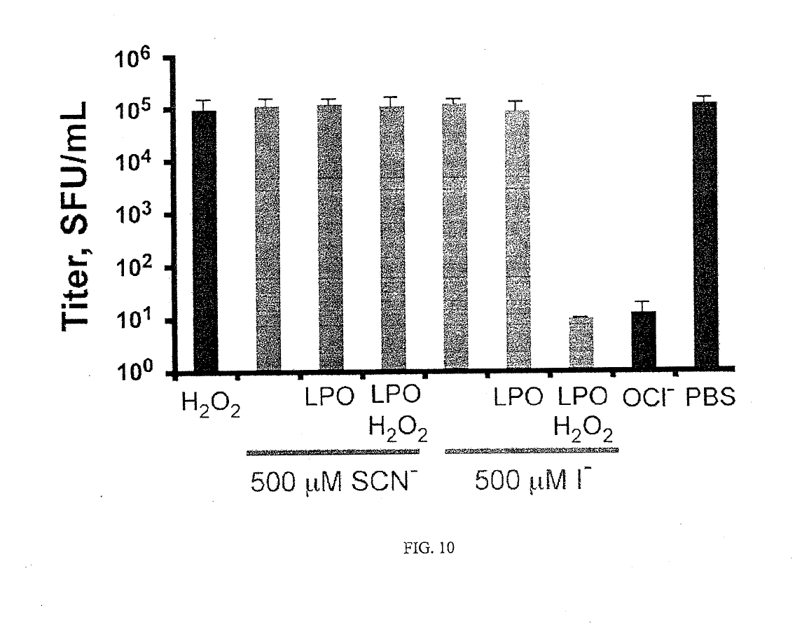

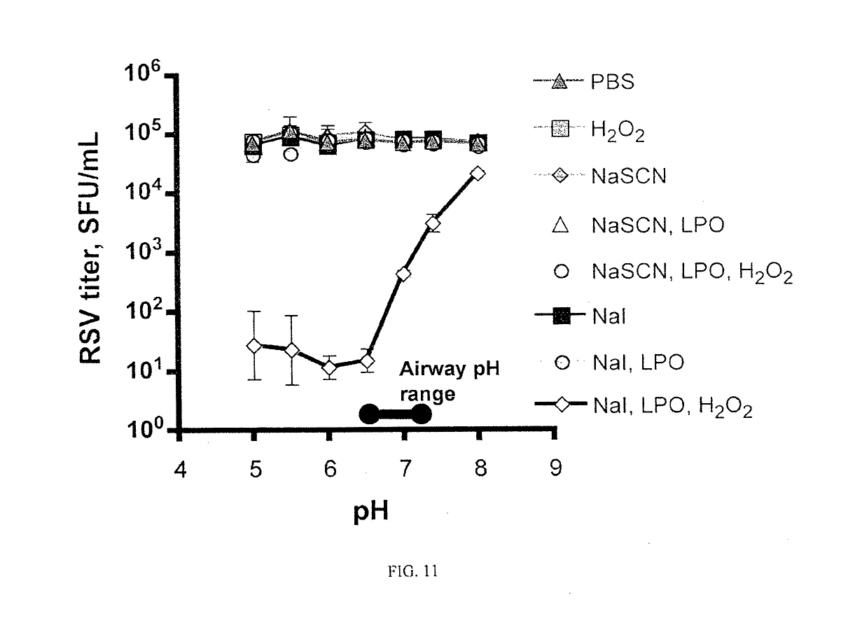

Respiratory Syncytial Virus (RSV): Results from cell free exposure of RSV to components of the Duox/LPO system with different halide substrates is shown in FIG. 10. As indicated, the virus was exposed to both OSCN⁻ and HOI or appropriate controls in PBS for 5 min, followed by serial dilution on Vero cells for titering. While OSCN⁻ exhibited minimal virucidal effects, HOI treatment of RSV caused a 5 log drop in titer (FIG. 10). In addition, the inventors investigated the effects of solution pH on the efficacy of hypohalides against RSV. As shown in FIG. 11, the antiviral effect of HOI was pH sensitive and most marked at pH≦6.5. This is of interest as the ASL pH has been reported to be in the range of ˜6.5-7.2 in health and disease. This knowledge aids in optimizing pH conditions for topical halide solutions.

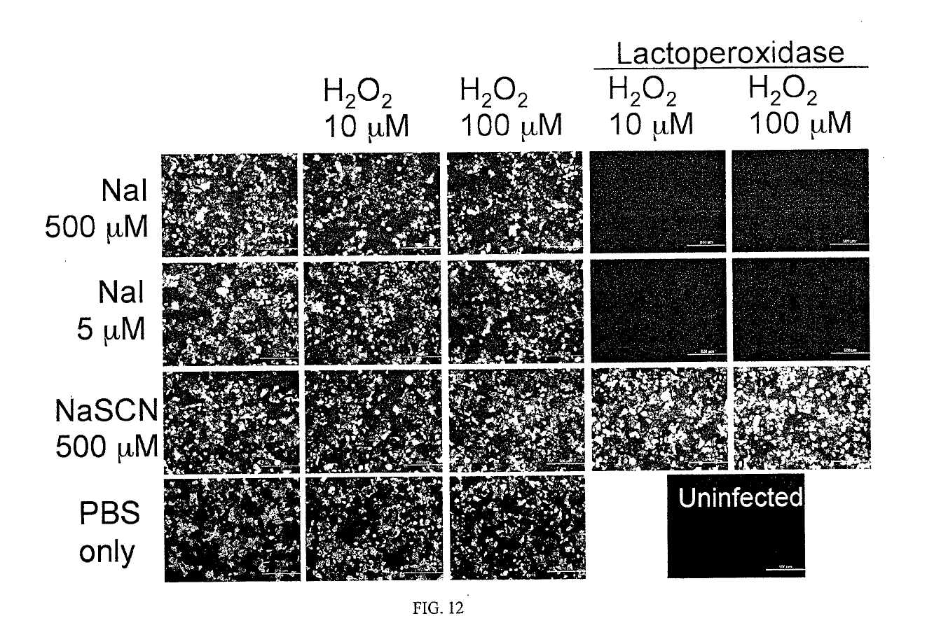

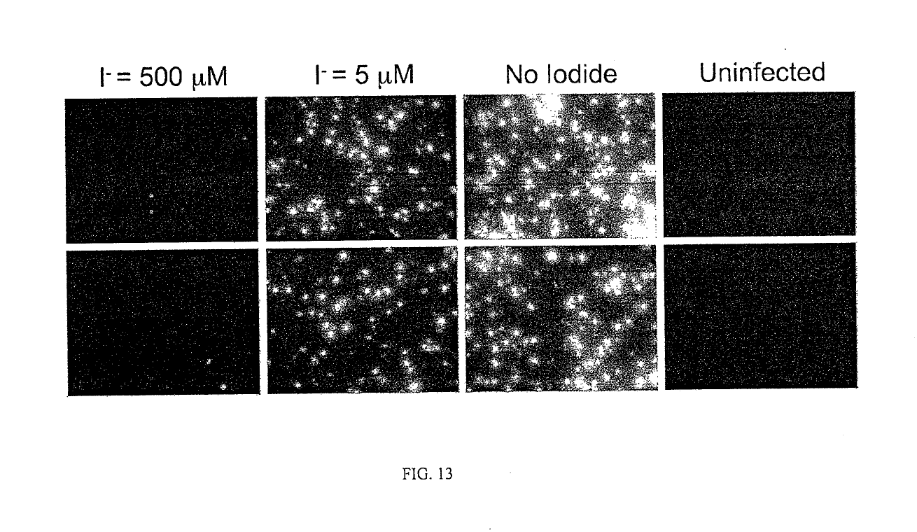

Adenovirus: The effects of OSCN⁻ and HOI on the viability of the encapsidated DNA virus adenovirus (serotype 5) were evaluated. The test adenovirus expresses the green fluorescent protein (eGFP) gene under control of the CMV promoter. Loss of eGFP expression is an indicator of virus inactivation. Under cell free conditions similar to those described in FIG. 10, adenovirus was inactivated by HOI but not OSCN⁻ (FIG. 12). The inventors also looked at virus inactivation in the setting of H₂O₂ production via epithelial Duox by adding LPO and the halide of interest to well differentiated airway epithelia. As shown in FIG. 13, airway epithelia provide sufficient H₂O₂ to support HOI, but not OSCN⁻ mediated inactivation of adenovirus.

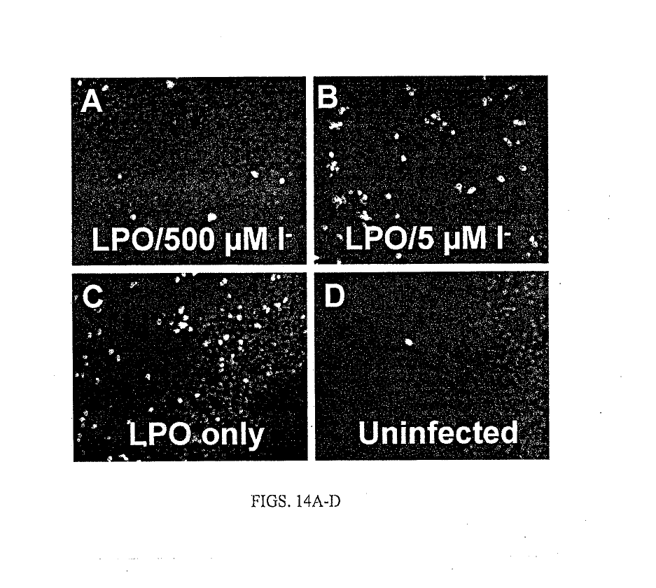

SARS Coronavirus: A similar approach to cell based killing was used to evaluate the efficacy of HOI against SARS-coronavirus. SARS-CoV (MOI 5) was applied to human primary epithelia under BSL3 containment in the presence of NaI and LPO as indicated. 24 hr later epithelia were immunostained for SARS-CoV N gene product. As shown in FIGS. 14A-D, airway epithelia provide sufficient H₂O₂ to support HOI mediated inactivation of SARS-CoV.

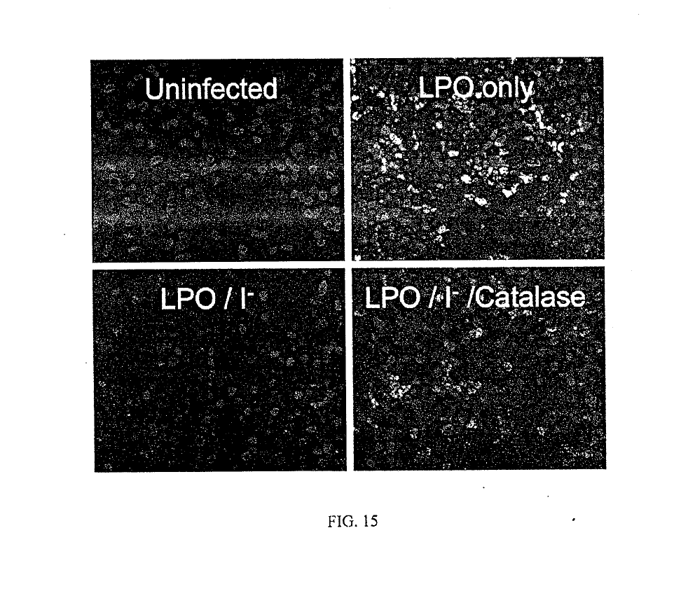

Influenza A: The inventors investigated the effects of OSCN⁻ or HOI on influenza A (H1N1, A/PR/8/34) on airway epithelia. Twenty MOI of influenza was added to the apical surface of primary air liquid interface cultures of human airway epithelia. Epithelia were treated with ATP (100 μM), 6.5 μg/ml LPO, and 500 μM NaSCN or NaI in a 50 μl of PBS, pH 6.5. En face confocal microscope images show a dose dependent decrease in viral antigen (NS1, green) was seen in the presence of OSCN⁻ (not shown) or HOI (FIG. 15). The antiviral effect was inhibited by catalase, indicating that it is H₂O₂ dependent.

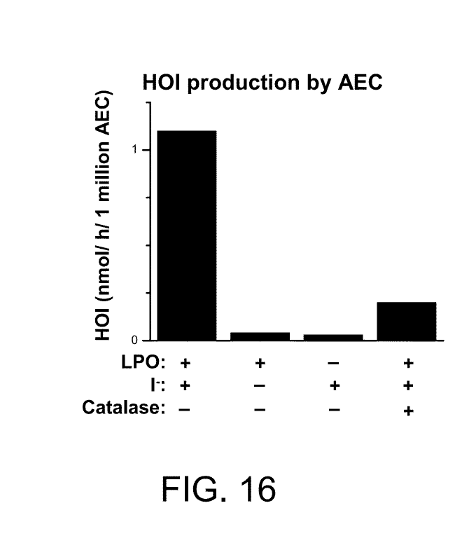

I⁻ enhances the antibacterial activity of the Duox/LPO enzymes. HOI is a more potent oxidant than OSCN⁻, but it is not toxic to eukaryotic cells at bactericidal concentrations (Vanden Abbeele, A., De Mee, H., Courtois, P., and Pourtois, M. 1996. Influence of a hypoiodite mouthwash on dental plaque formation in vivo. Bull Group Int Rech Sci Stomatol Odontol. 39:57-61). Since I⁻ is a high-affinity substrate for LPO, the Duox/LPO enzymes might generate HOI even in the presence of SCN⁻. Thus, the inventors examined the HOI-producing capacity of airway epithelial cells (AEC) and the potential consequences of HOI production for the host defense. As indicated, iodination of fluorescein shifts the absorption maximum of this dye from 488 nm to 508 nm (Slungaard, A., and Mahoney, J. R. J. 1991. Thiocyanate is the Major Substrate for Eosinophil Peroxidase in Physiologic Fluids. Implications for cytotoxicity. J Biol Chem 266:4903-4910), and the inventors have verified that: (i) fluorescein reacts with HOI, leading to an increased absorbance at 508 nm, and (ii) fluorescein does not react with H₂O₂ and OSCN⁻. Also, the DTNB assay for OSCN⁻ production does not detect HOI. Next, the inventors tested the HOI-producing capacity of AEC cultures. Differentiated human AEC were cultured on transparent Transwell filters and stimulated with apical ATP (100 μM) to maximize H₂O₂ production. In addition to ATP, the apical buffer contained I⁻ (400 μM), LPO (9 μg/ml), and fluorescein (16 μM). Negative control samples either lacked both LPO and I⁻, or contained catalase (1000 U/ml). Changes in absorbance at 508 nm were measured every 30 min over a course of 3 hours (FIG. 16). These experiments suggest that AEC can support HOI production if I⁻ is topically applied to the mucosal surface, and that the inventors can detect HOI specifically.

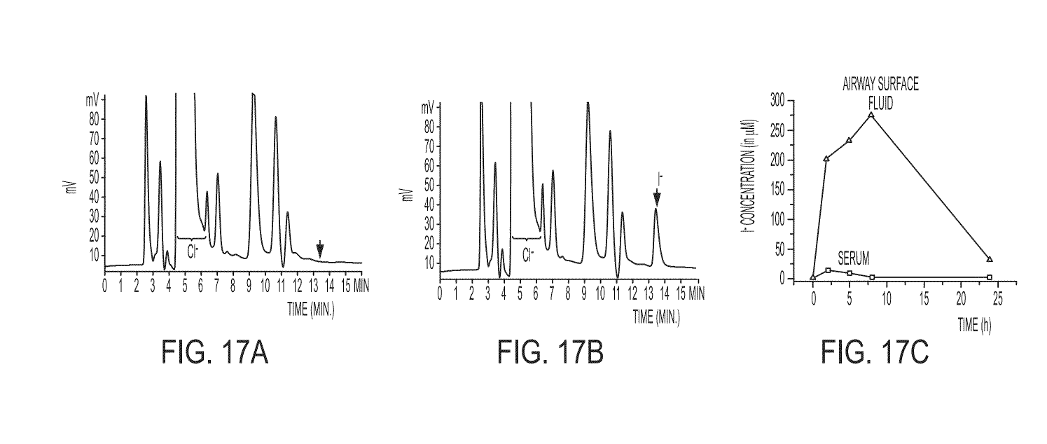

Oral iodine accumulation in airway secretions. The inventors decided to study the I⁻ concentration in the conducting airways of sheep following oral KI administration. One trial has been performed. Nasal airway fluid was harvested from a healthy volunteer using a microcapillary probe, and blood was drawn from an arm vein. Following verification of normal thyroid function (based on blood TSH, and free T4 levels), the subject swallowed a commercially available FDA-approved KI tablet (Iosat, 130 mg KI). 2, 5, 8, and 24 hours later, nasal fluid and blood samples were collected again, and all samples were analyzed for I⁻ concentration, using anion-exchange chromatography. Before KI intake, the I⁻ content of the airway surface fluid was below the detection limit of the inventors' assay (0.5 μM). However, two hours after KI intake, I⁻ accumulated in the airway surface fluid at ˜200 μM. Moreover, this high I⁻ concentration was maintained for several hours and exceeded the serum I⁻ level by more than 50-fold at 8 hours (FIGS. 17A-C). Thus, oral intake of 130 mg KI leads to airway surface fluid accumulation of I⁻ at levels that—based on the inventors' cell culture assays—can support antibacterial and antiviral activities. Iodine administration has been used commonly in sheep and cattle for deep-seated bacterial infections (but not respiratory infections) with minimal side-effects.

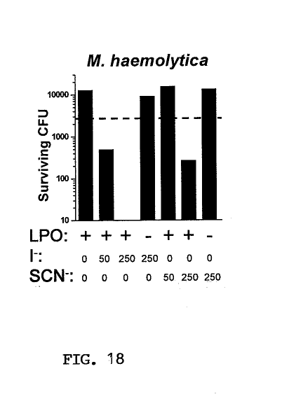

Antimicrobial activity of hypohalides against a major veterinary pathogen. The inventors proceeded to compare the antibacterial activities of the oxidative system in the presence of different I⁻ and SCN⁻ concentrations. For these bacterial killing assays, the inventors used two human airway pathogens (S. aureus and NTHi) and one sheep airway pathogen (M. haemolytica). Whereas higher SCN⁻ concentration than 120 μM was necessary for the killing of S. aureus on AEC cultures, the presence of 50 μM I⁻ supported the complete elimination S. aureus by AEC (data not shown). Notably, the I⁻-dependent bacterial killing activity was inhibited by the H₂O₂ scavenger catalase and required LPO, which indicates the oxidative nature of the antibacterial mechanism. The greater potency of I⁻ versus SCN⁻-dependent bacterial killing was even more evident when NTHi was tested. NTHi was resistant to OSCN⁻, whereas 50 μM I⁻ was already sufficient to support a strong NTHi-killing activity of AEC in the presence of LPO and in the absence of catalase (data not shown). M. haemolytica was susceptible for both the SCN⁻ and I⁻ supported antibacterial activities, but the elimination of this bacterium was more significantly more effective in the presence of I⁻ as compared to SCN⁻ (FIG. 18).

All of the compositions and methods disclosed and claimed herein can be made and executed without undue experimentation in light of the present disclosure. While the compositions and methods of this invention have been described in terms of preferred embodiments, it will be apparent to those of skill in the art that variations may be applied to the compositions and methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### V. REFERENCES

The following references, to the extent that they provide exemplary procedural or other details supplementary to those set forth herein, are specifically incorporated herein by reference.
U.S. Pat. No. 4,321,251
U.S. Pat. No. 4,919,918
U.S. Pat. No. 5,158,761
U.S. Pat. No. 5,250,298
U.S. Pat. No. 5,372,801
U.S. Pat. No. 5,470,561
U.S. Pat. No. 5,503,853
U.S. Pat. No. 5,681,827
U.S. Pat. No. 5,695,746
U.S. Pat. No. 5,830,499
U.S. Pat. No. 5,993,785
U.S. Pat. No. 6,086,852
U.S. Pat. No. 6,117,417
U.S. Pat. No. 6,165,494
U.S. Pat. No. 6,165,615
U.S. Pat. No. 6,166,004
U.S. Pat. No. 6,166,043
U.S. Pat. No. 6,166,084
U.S. Pat. No. 6,171,611
U.S. Pat. No. 6,348,187
U.S. Pat. No. 6,348,502
U.S. Pat. No. 6,387,352
U.S. Pat. No. 6,503,481
U.S. Pat. No. 6,551,578
U.S. Pat. No. 6,555,376
U.S. Pat. No. 6,610,272
U.S. Pat. No. 6,702,998
U.S. Pat. No. 6,797,704
U.S. Pat. No. 6,841,539
U.S. Patent Appn. 20020044910
U.S. Patent Appn. 20030152530
U.S. Patent Appn. 20040076590
U.S. Patent Appn. 20040146919
U.S. Patent Appn. 20050143336
U.S. Patent Appn. 20050196343
U.S. Patent Appn. 20050260276
Ackermann and Brogden, Microbes. Infect., 2(9):1079-1088, 2000.
Banfi et al. Am. J. Respir. Crit. Care Med., 175(9):967, 2007.
Belding et al., Science 167: 195-196, 1970.
Conner et al., Am. J. Respir. Crit. Care Med., 166(12 Pt 2):557-61, 2002.
Conner et al., FEBS Lett., 581(2):271-278, 2007.
Czuprynski et al., Anim. Health Res. Rev., 5(2):277-282, 2004.
El-Chemaly et al., Am. J. Respir. Crit. Care Med., 167(3):425-430, 2003.
EP 0 361 908
Fauci, Nature, 435:423-424, 2005.
Forteza et al., Am. J. Respir. Cell Mol. Biol., 32(5):462-469, 2005.
Fragoso et al., J. Physiol., 561(Pt 1):183-194, 2004.
Horimoto et al., Nat. Rev. Microbiol., 3:591-600, 2005.
Idson, In: Pharmaceutical Dosage Forms, Disperse Systems, Lieberman et al. (Eds.), Marcel Dekker, NY, 1:199-243, 1988.
International Cosmetic Ingredient Dictionary and Handbook, 10^{th} Ed., 2004.
LaForce et al., Am. J. Prey. Med., 10(Suppl):31-44, 1994.
Moskwa et al., Am. J. Respir. Crit. Care Med., 175:174-183, 2007.
Palese, Nat. Med.,10:S82-7, 2004.
Pedemonte et al., J. Immunol., 178(8):5144-5153, 2007.
Remington's Pharmaceutical Sciences” 15^{th} Ed., pages 1035-1038 and 1570-1580, 1990.
Salathe et al., Am. J. Respir. Cell Mol. Biol., 17(1):97-105, 1997.
Wijkstrom-Frei et al., Am. J. Respir. Cell Mol. Biol., 29(2):206-212, 2003.

1. A method of treating or preventing a viral infection in a subject comprising administering a therapeutically effective amount of a halide.

2. The method of claim 1, wherein administering comprises inhalation, topical administration, oral administration or systemic administration.

3. (canceled)

4. The method of claim 1, wherein said viral infection is of the lung and/or respiratory system.

5. The method of claim 1, wherein said subject is a human that suffers from cystic fibrosis.

6. The method of claim 1, wherein said halide is iodide or a potassium or sodium salt thereof.

7. The method of claim 1, wherein said viral infection is caused by respiratory syncytial virus, influenza virus, adenovirus, measles virus, arenavirus, filovirus, echovirus, parainfluenza virus, rhinovirus, Coxsackie virus, Epstein Barr virus, or cytomegalovirus.

8. The method of claim 1, wherein said viral infection is caused by a coronavirus or herpesvirus.

9. The method of claim 1, further comprising administering lactoperoxidase, myeloperoxidase, horseradish peroxidase or an anti-viral drug to said subject.

10. The method of claim 1, wherein treating comprises limiting the duration or severity of symptoms, limiting viral replication, decreasing viral load or increasing viral clearance.

11. A method of treating or preventing a lung/respiratory pathogen infection in a subject comprising administering a therapeutically effective amount of a halide.

12. The method of claim 11, wherein said respiratory pathogen is a bacterium or fungus.

13. The method of claim 12, wherein said bacterium is H. influenzae or S. aureus.

14. The method of claim 11, further comprising administering lactoperoxidase, myeloperoxidase, horseradish peroxidase or an antibiotic.

15. The method of claim 11, wherein treating comprises decreasing the bacterial load.

16. The method of claim 11, wherein administering comprises inhalation, topical administration, oral administration or systemic administration.

17-18. (canceled)

19. The method of claim 11, wherein said subject is a human that suffers from cystic fibrosis.

20. The method of claim 11, wherein said halide is iodide or a potassium or sodium salt thereof.

21. A method of enhancing endogenous respiratory antiviral defense in a subject comprising administering a therapeutically effective amount of a halide.

22. An inhaler device that delivers a unit dose comprising a therapeutically effective amount of halide or halide salt in a liquid or aerosol carrier.

23. The inhaler device of claim 22, wherein said halide or halide salt comprises iodine, or a potassium or sodium salt thereof.
